(19) United States
(12) Patent Application Publication
(10) Pub. No.: US 2006/0204443 A1
(43) Pub. Date: Sep. 14, 2006

(54) Methods for tumor treatment using dendrimer conjugates

(75) Inventors: Hisataka Kobayashi, Rockville, MD (US); Peter L. Choyke, Bethesda, MD (US)

Correspondence Address: KLARQUIST SPARKMAN, LLP, 121 S.W. SALMON STREET, SUITE #1600, PORTLAND, OR, 97204-2988, US

(73) Assignee: The Government of the USA as represented by the Secretary of the Dept. of Health & Human Services

(21) Appl. No.: 11/371,780

(22) Filed: Mar. 9, 2006

Related U.S. Application Data
(60) Provisional application No. 60/661,107, filed on Mar. 11, 2005.

(51) Int. Cl. A61K 49/10; A61N 5/00; A61K 31/765

(52) U.S. Cl. 424/9.32; 424/78.27; 600/1

(57) ABSTRACT

Methods are disclosed for treating a tumor. A dendrimer conjugate is administered to a subject having a tumor. The dendrimer of the dendrimer conjugate is a generation 5 DAB, generation 2 polylysine, or generation 6-8 PAMAM dendrimer. The dendrimer conjugate comprises an effective amount of an anti-tumor agent. The anti-tumor agent is selectively concentrated in the lymphatic system to treat metastatic disease. In certain examples, the anti-tumor agent is an activatable anti-tumor agent and is activated once the anti-tumor agent is selectively concentrated in the lymphatic system.

### CROSS REFERENCE TO RELATED APPLICATION

This application claims priority of, and incorporates by reference, U.S. Provisional Patent Application No. 60/661,107, filed Mar. 11, 2005.

### FIELD

Methods of treating neoplastic tissue or metastatic cells are disclosed. More specifically, the disclosure relates to dendrimer conjugates that are useful for targeting anti-tumor agents to tumor tissue, particularly in the lymphatic system.

### BACKGROUND

The lymphatic system is the network of circulatory vessels or ducts in which the interstitial fluid bathing the cells of all tissues (except nerve tissue) is collected and carried to join the cardiovascular system. The lymphatic system is of importance in transporting digested fat from the intestine to the bloodstream, in removing and destroying foreign substances, and in resisting the spread of pathogens throughout the body. Lymphatic capillaries are more permeable than ordinary blood capillaries, so molecules too large to directly enter the blood stream will pass into the lymphatic system for transport away from tissues. The lymphatic capillaries merge to form larger ducts that intertwine about the arteries and veins. The lymph fluid in these larger ducts is transported by the muscular movements of the body, and reverse flow is avoided by one-way valves located at intervals along the lymph vessels.

Interposed along the course of the lymphatic vessels are lymph nodes (LNs), which are bean-shaped organs containing large numbers of leukocytes embedded in a network of connective tissue. All the lymph fluid flowing through the lymphatic system to the bloodstream must pass through several of these nodes, which filter out bacteria, viruses, tumor cells, toxins and other pathogenic material for sequestration or removal from the body. The nodes serve as a center for the production of phagocytes, which engulf bacteria and other pathogens. During the course of any infection, the nodes become enlarged because of the large number of phagocytes being produced. Since certain malignant tumors also tend to spread through the lymphatic system, surgical removal of all nodes that are suspected of being involved in the spread of such malignancies is an accepted but undesirable therapeutic procedure.

Sentinel node biopsy is a technique used to determine more accurately whether a cancer has spread (metastasized), or is localized to a primary tumor. The “sentinel” lymph node (SLN) is the first lymph node along one or more paths of lymphatic drainage away from the primary tumor before lymphatic flow drains secondarily into the remaining regional LNs. A negative biopsy and analysis of the SLN(s) for metastatic cells reliably indicates that a cancer has not metastasized, and may spare a cancer patient more drastic treatments and procedures. However, identifying and locating the SLN can be difficult, and prior to more recent methods for identifying the SLN, all of the regional LNs near a tumor were removed for analysis by a pathologist. In many patients, the regional LNs turned out to be free of tumor cells, but these patients were still placed at risk of developing the potential complications of major LN resection, including chronic swelling, discomfort, reduced mobility, and increased risk of infection. The SLN concept has been applied successfully to the treatment of a variety of cancers, including cancer of the penis, skin, breast, vulva, lung, head and neck (including papillary thyroid carcinoma).

Breast cancer is the most common malignancy in women in the United States, resulting in approximately 45,000 deaths annually. Landis et al., CA Cancer J. Clin.; 49: 8-31, 1999. The presence of lymph node metastases has major negative prognostic implications in breast cancer patients, and is the major criterion for determining the need for adjuvant chemotherapy. For many years, surgical dissection of the axillary lymph nodes was used to assess lymph node involvement by breast cancer. Now, at least two accepted methods exist for identifying and locating sentinel lymph nodes associated with breast and other types of cancer.

The commonly used methods for identifying and locating the SLN employ peritumoral injections of either isosulfan blue dye, or a radionuclide-labeled sulfur or albumin colloid (radiocolloid). The dye or radiocolloid serves as a tracer of lymphatic flow away from a tumor. In the blue dye technique, the SLN is detected by direct visualization, which requires blind dissection of tissue until the “dyed” SLN is detected. In the radiocolloid technique, the SLN is located based on a localized accumulation of radioactivity that is detected using a hand-held gamma ray counter. Alazraki et al., Update on Nuclear Medicine; 39: 947-956, 2001. The radionuclide method can assist in localization of the SLN, but it has poor spatial resolution. Therefore, the surgeon still has to search through tissue to locate the SLN.

The dye and radionuclide methods may be combined, with the radionuclide used to find the general area of the SLN and the dye used to help the surgeon locate the exact position of the SLN within that general area. Still, a LN with high radioactivity and/or intense blue staining is not necessarily a SLN since the radiocolloids and blue dye tend to move away from the actual SLN to more distant LNs during the procedures. A “first appearance criterion” has been applied to identify a SLN as a node that is first in time to receive a dye or radiocolloid that has been injected into or near a tumor. However, the dye and radiocolloid methods offer insufficient temporal resolution to assure reliable SLN identification based on this criterion.

Magnetic resonance imaging (MRI) has been proposed as a method for identifying SLNs based on a first appearance criterion, and a number of magnetic resonance contrast agents have been tried for lymphangiography (visualization of the lymph system and lymph flow therein). For example, the low molecular weight contrast agent gadopentetate dimeglumine (GPDM) has been used to image lymph flow. Suga et al., Acta Radiologica; 44: 35-42, 2003. GPDM does not, however, exhibit lymphotropic properties, and the lymphatic distribution of this compound appears to be unpredictable and inconsistent enough to preclude its use in a clinical setting.

Ultra-small iron oxide particles (USPIO) and Gadomer-17 have been used as contrast agents for lymphangiography. In the case of USPIO, it has been reported that MRI with this contrast agent does not permit observation of small lymph nodes. Hoffman et al., Laryngoscope; 110: 1425-1430, 2000. Furthermore, since USPIO provides negative contrast of lymph nodes relative to surrounding tissue, it is not compatible with image-guided dissection or biopsy of lymph nodes. The use of Gadomer-17 contrast agent has been somewhat more successful for locating and identifying SLNs, and for enabling image-guided procedures. Torchia et al., J. Surgical Oncology; 78: 151-156, 2001. Nonetheless, neither of these MRI methods provides images of lymphatic system structure with sufficient spatial resolution to permit direct non-invasive assessment of disease states in the lymphatic system (i.e. without biopsy).

Prior treatments for cancers involving the lymphatic system typically involve traditional radiation therapy and/or chemotherapy. Traditional radiation therapy and chemotherapy can have a number of undesirable side effects and often affect both healthy tissue and neoplastic tissue. In addition, some conditions, such as certain types of non-Hodgkin's lymphoma, are resistant to conventional treatment.

It is known that targeted radiation therapy, including neutron capture therapy (NCT), has the potential for treating various types of cancers. These therapies have the potential to present a number of benefits as compared to existing methods of cancer treatment, such as surgical procedures, chemotherapy, and conventional radiation treatment. Although surgical removal of tumors typically only involves the specific tissue to be removed, accessing that tissue can be invasive and difficult. Furthermore, surgical excision may not remove all malignant tissue or may result in malignant tissue contaminating another area of the patient's body. Chemical and radiation treatments may be non-selective, targeting both healthy and neoplastic tissue or metastatic cells, and may have a number of undesirable side effects.

NCT has been shown to be effective in treating cancers which are normally resistant to radiotherapy, including lymphoma and skin cancer. NCT may also be useful in treating cancers that are not clearly defined. Since neutrons have no charge, a neutron beam can penetrate normal tissue with minimal radiation side effects, while potentially depositing almost all its energy within the target.

Many targeted radiation therapies, including NCT, involve a two step process. First, a radiation absorbing agent, such as a compound complexed to an atom (a neutron capture element or “NCE”) having a large radiation capture cross section, is introduced into a subject. The radiation absorbing agent selectively associates in vivo with neoplastic tissue or metastatic cells, as opposed to healthy cells. The target area of the patient is then irradiated, such as with a beam of neutrons. Upon absorption of the radiation, the NCE produces energetic particles, for example by fission of the NCE. These byproducts, which may include alpha particles, recoil atoms, and Auger electrons, often have high energy and high linear energy transfer and may kill or damage nearby target cells.

Boron and gadolinium have been used in NCT as NCEs. In NCT, NCEs are typically atoms having a large neutron capture cross section and which emit energy when bombarded with neutrons. For example, ¹⁰B has been used as a NCE in a neutron capture agent (NCA). ¹⁰B has a high neutron capture cross section, 3,840 barn, compared to elements of healthy tissue, the components of which typically have neutron capture cross sections which are orders of magnitude lower.

The NCE may be complexed to a molecule that aids in transporting the NCE target to the treatment site, to form an NCA. After the NCA is at the treatment site, the patient is exposed to a beam of neutron containing radiation, such as a beam directed at the target tissue.

The neutron radiation is absorbed by the NCE, but is barely absorbed by healthy tissue. Upon absorption of a neutron, ¹⁰B undergoes fission to form a 0.84 MeV lithium nucleus (⁷Li) and to emit a 1.47 MeV alpha particle. These high energy particles are efficient at killing local cells, but have a range of only 7.3 and 4.0 μm, respectively, which is less than the typical 10 μm diameter of a cell. The short range and high energy of the ¹⁰B fission products make them very specific for affecting neoplastic cells, while sparing healthy tissue.

However, there have been problems in the clinical use of boron NCT. Because of the short range of the emitted particles, boron containing NCAs must be highly concentrated in the tissue of interest. However, there have been difficulties in preparing boron containing NCAs that may be administered in sufficient quantities to provide the necessary in vivo NCA concentration to effectively kill neoplastic cells.

In addition to boron NCT, gadolinium based NCT has been proposed. There are two Gadolinium isotopes that are useful for NCT. The ¹⁵⁷Gd isotope has a natural abundance of 15.7% and a neutron capture cross section of about 254,000 barn. Another isotope, ¹⁵⁵Gd, has a neutron capture cross section of about 61,000 barn.

After neutron absorption, ¹⁵⁷Gd emits gamma radiation and forms ¹⁵⁸Gd. In addition, the rearrangement of electrons in the gadolinium nucleus produces Auger electrons having high linear energy transfer, although they typically have a more limited range than the alpha particles produced by ¹⁰B. Auger electrons, having energies typically less than about 41 keV, are known to damage DNA.

Although experiments have been performed to study the viability of Gd-NCT as a clinical therapy, an ideal gadolinium NCA has not yet been found. A major current limitation of Gd-NCT is that about 400 ppm of natural Gd(III), which equates to about 64 ppm of Gd-157, is used for efficient cell killing. This concentration is very difficult to achieve using conventional intravenous routes of delivery. Therefore, most in vivo studies have been performed with intra-tumoral or intra-arterial injection of agents.

Another problem with many gadolinium NCAs is that they may not be sufficiently retained in vivo for NCT to be conducted. For example, Magnevist, a commercial gadolinium containing contrast agent, was found to clear too rapidly from the body to be used as a NCA. Tokumitsu et al., Cancer Lett.; 150:177-182, 2000.

### SUMMARY

Methods are disclosed for treating a tumor by administering to a subject having a tumor a dendrimer conjugate comprising an effective amount of an anti-tumor agent. The dendrimer portion of the conjugate is a generation 5 DAB dendrimer, a generation 2 polylysine dendimer, or a generation 6-8 PAMAM dendrimer. In particular examples, the dendrimer comprises a generation 6 PAMAM dendrimer, such as PAMAM-G6. The anti-tumor agent is then selectively concentrated in the lymphatic system by the dendrimer, to effectively treat the tumor (such as metastatic or micro-metastatic disease) present in the lymphatic system (for example in the lymph nodes). In a certain example, the anti-tumor agent is an activatable anti-tumor agent which may be activated once the anti-tumor agent has been selectively concentrated in the lymphatic system. The activatable anti-tumor agent is gadolinium in certain examples. When an activatable anti-tumor agent is used, it may be activated by applying physical energy to the subject's body, for example by external application of that energy to the body. In particular examples, the external energy is heat, ultrasound, or electromagnetic energy. In a particular example, the physical energy is a particle beam, such as a neutron beam.

The dendrimer conjugates may include an imaging agent, which permits the lymphatic system to be imaged when selective intra-lymphatic concentration of the dendrimer occurs. Further, when the dendrimer conjugate includes an activatable anti-tumor agent, the method may include selectively applying physical energy to the subject's body to selectively activate the anti-tumor agent in the lymphatic system. In some disclosed examples, the dendrimer conjugate includes gadolinium, and the gadolinium acts as a contrast agent to image the lymphatic system.

In a particular example, the dendrimer conjugate includes a gadolinium imaging agent that is activatable by a neutron beam. Once the gadolinium containing dendrimer conjugate is concentrated in the lymphatic system, the lymphatic system is imaged by detecting selective concentration of the dendrimer conjugate in the lymphatic system. The presence of tumor in lymph nodes can also be detecting using this imaging technique. A neutron beam is then selectively applied to the imaged lymphatic system to selectively activate the anti-tumor agent at target areas in the lymphatic system for the treatment of metastatic tumor. In this example, the target area may be a lymph node, such as a sentinel lymph node, or a lymphatic vessel. The target area, when imaged, may show evidence of primary or metastatic tumor.

The disclosed methods therefore permit delivery of anti-tumor agents to the lymphatic system in sufficient concentrations to have a desired anti-tumor effect. It also permits the non-surgical delivery of the agents to the target site, although intra-operative delivery is possible.

### BRIEF DESCRIPTION OF THE DRAWINGS

FIG. 1 is a schematic representation of the general structures of lower generation DAB-Am and PAMAM dendrimers; higher generation DAB-Am and PAMAM dendrimers have similar structures, but are larger with additional branches and terminal amino groups. As shown, a doubling of the branches and the number of terminal amino groups occurs with each successively higher generation.

FIG. 2 is a schematic drawing illustrating the potential dual roles of ¹⁵⁷Gd as a neutron capture element and as an imaging agent. As shown on primarily the left side of FIG. 2, a ¹⁵⁷Gd isotope can capture neutrons due to its large capture cross section (CCS) resulting in an emission of a Beta particle (an Auger electron) for therapy. Additionally, as shown on the right side of FIG. 2, ¹⁵⁷Gd is paramagnetic and can act as an enhancer of T1 relaxation leading to increased signal on T1 weighted MRI.

FIG. 3 is a schematic drawing illustrating the mechanism of interstitial delivery of nano-sized particles. The top portion of FIG. 3 illustrates that interstitial injection of G6 dendrimers results in specific uptake by the lymphatics resulting in complete opacification of the lymph nodes (black). The bottom portion of FIG. 3 illustrates that smaller contrast agents are absorbed by the lymphatics, but leak out from them by convection around the tumor, resulting in lower lymph node concentrations (gray).

FIG. 4 is a magnetic resonance image and a schema of the imaged structures in the region of the mammary gland that was obtained 36 min after injection of a PAMAM-G6 dendrimer conjugate.

FIG. 5 is a series of 3D dynamic mammo-lymphangiograms obtained following the sequential injection of GPDM and the PAMAM-G6 contrast agent (approximately 36 minutes later; see the time course inset) showing the lack of enhancement of lymphatic structures in the absence of the PAMAM-G6 agent, and the remarkable image contrast obtained for the lymphatics draining the mammary gland following administration of the PAMAM-G6 agent.

FIG. 6A is set of 2D-fastIR stereo-view images of a BALB-neuT transgenic mouse bearing a bilateral breast tumor (solid arrows) and two metastatic tumors (broken arrows) in the axilla and the lateral chest wall that was obtained following administration of the PAMAM-G6 contrast agent. A schema of the images also is shown to aid in the interpretation of the images.

FIG. 6B is a series of 3D dynamic mammo-MR-lymphangiograms obtained for the same mouse as shown in FIG. 6A that was obtained following administration of the PAMAM-G6 contrast agent. Several dilated lymphatic vessels extending from the breast tumor to two tumors in lymph nodes at the lateral chest wall were clearly imaged.

FIG. 7A is a set of 2D-fastIR stereo-view images of a mouse with a PT-18 tumor (solid arrow) in the breast and a tumor (broken arrow) in the axillary lymph node obtained following administration of the PAMAM-G6 contrast agent.

FIG. 7B is a series of 3D mammo-MR-lymphangiograms of the same mouse shown in FIG. 7A showing that the axillary lymph node tissue with metastatic tumor cells did not show enhancement by the PAMAM-G6 contrast agent. However, the lymphatic vessel flowing into the lymph node with a metastatic tumor was dilated and showed enhancement. A schema to aid interpretation of the images is also shown as an inset.

FIG. 8A is a pair of 3D mammo-MR-lymphangiograms of axillary lymph nodes without (left image) and with (right image) a PT-18 metastatic tumor showing, with surprising detail, the lack of filling of the metastatic lymph node and dilation of the afferent lymph vessel of the lymph node.

FIG. 8B is a pair of histological sections (hematoxylin-eosin stained) confirming tumor growth in the non-enhanced portion of the metastatic lymph node (right-hand section, corresponding to the right-hand image of FIG. 8A) compared to the normal lymph node which showed no filling defects (left-hand section, corresponding to the left-hand image of FIG. 8A).

FIG. 9 shows, on the left, a typical 3D-micro-MR lymphangiogram of normal mice taken 45 minutes after administration of the PAMAM-G8 contrast agent. A schema that aids in interpretation of the MR image is shown on the right. The injection site and locations of the components of the deep lymphatic system are indicated by the labeled arrows.

FIG. 10A is a set of whole-body 3D-micro-MR MIP images of mice injected intracutaneously in all four middle phalanges with 0.005 mmolGd/kg of PAMAM-G8, DAB-G5, PAMAM-G4, Gadomer-17 or GPDM taken at 10 minutes after injection, showing the superior image detail obtained with the disclosed dendrimer contrast agents in comparison to both Gadomer-17 and GPDM.

FIG. 10B is a set of whole-body 3D-micro-MR MIP images of the same mice imaged in FIG. 10A, only at 45 minutes after injection, showing the persistent superior image detail obtained with the disclosed dendrimer contrast agents in comparison to both Gadomer-17 and GPDM.

FIG. 11A is a graph showing the axillary lymph node-to-muscle image signal intensity ratio for PAMAM-G8, DAB-G5, PAMAM-G4, Gadomer-17, and GPDM over time following administration of each of the contrast agents. The values are expressed as the mean and the standard deviation (N=5 or 6). The asterisks indicate significant differences from the group with PAMAM-G8 (P<0.01).

FIG. 11B is a graph showing the axillary lymph node-to-liver image signal intensity ratios for PAMAM-G8, DAB-G5, PAMAM-G4, Gadomer-17, and GPDM over time after administration of each of the contrast agents. The values are expressed as the mean and the standard deviation (N=5 or 6). The asterisks indicate significant differences from the group with PAMAM-G8 (P<0.01).

FIG. 12 is a pair of whole-body dynamic 3D-micro-MR lymphangiograms of a mouse with Concanacalin A lymphangitis that was injected intracutaneously into all four middle phalanges with 0.005 mmolGd/kg of PAMAM-G8, and imaged at 10 and 45 minutes following administration of the contrast agent.

FIG. 13 is a pair of whole-body 3D-micro-MR lymphangiograms of IL-15 transgenic mice (high producer) with induced lymphoadenopathy and subcutaneous involvement of lymphoproliferative disorder that were obtained 45 minutes after administration of (left) 0.005 mmolGd/kg of PAMAM-G8 and (right) DAB-G5. Dilation of subcutaneous lymphatic vessels (broken arrows) and swollen right axillary lymph nodes (solid arrows) are indicated on the images.

FIG. 14A is a whole-body 3D-micro-MR lymphangiogram of a mouse with a lymph node metastasis obtained 45 minutes after injection of 0.005 mmolGd/kg of PAMAM-G8. Large inguinal and abdominal tumors (asterisks) accompanied by the left inguinal lymph node (long arrow) are shown, along with dilated lymphatic vessels surrounding the tumor and a collateral lymphatic vessel, which communicated with the thoracic duct via the axillary lymph node (arrowheads).

FIG. 14B is a whole-body 3D-micro-MR lymphangiogram of a mouse with a subcutaneously xenografted tumor of MC-38 cells that was obtained 45 minutes after injection of 0.005 mmolGd/kg of PAMAM-G8. A large inguinal tumor (astersisk) is shown accompanied by the left normal inguinal lymph node (long arrow). No dilated lymphatic vessels surrounding the tumor are seen.

FIGS. 15A and 15B are whole-body 3D-micro-MR lymphangiograms of normal mice given intracutaneous injections into all four middle fingers with 0.05 mmolGd/kg of PAMAM-G8 and GPDM, respectively.

FIG. 16A is a pair of whole-body 3D micro-MR-lymphangiograms (stereo view) of a mouse with concanavilin-A-induced lymphangitis showing dilated lymph vessels (arrows).

FIG. 16B is a two-dimensional micro-MR image of the liver of the mouse shown in FIG. 16A having concanavalin-A lymphangitis, showing enhancement adjacent to the vascular structures (arrows), which did not show enhancement.

FIG. 16C is a histological microscope picture (20×) of the region of the mouse's liver shown in FIG. 168B showing that lymphocytes had mainly infiltrated adjacent to the vascular structures (arrows) in the same place where enhancement was shown in FIG. 16B.

FIG. 17A is a composite of a whole-body 3D-micro-MR and neck and pelvic 2D micro-MR lymphangiograms of a mouse with L-15 transgenic-induced lymphoadenopathy with CD8⁺ T-cells taken 45 minutes after intracutaneous injection of PAMAM-G8 into the fingers of the mouse showing enlargement and a lack of enhancement within several of the lymph nodes.

FIG. 17B is a microscopic picture (20×) of an enlarged lymph node obtained from the IL-15 transgenic mouse in FIG. 17A, showing that the germinal center structure of the lymph node was no longer seen and was replaced by a homogeneous dense infiltration of lymphoid cells.

FIGS. 18A-18C are composites of three 3D-micro MR images of the external iliac lymph nodes in normal mice (18A), in nude mice that spontaneously develop oral ulcers and urinary tract infections (18B), and in L-15 transgenic mice with lymphoproliferative or neoplastic disease (18C). These images were taken 45 minutes after administration of PAMAM-G8 and demonstrate that the disclosed methods can be used to differentiate infectious and neoplastic changes in the lymph nodes.

FIG. 19A is a series of 3D dynamic MR lymphangiograms obtained 12 minutes post-injection of various PAMAM dendrimer based contrast agents. The G6 contrast agent of 9 nm in diameter depicted both lymph nodes and lymphatic vessels most efficiently among all agents examined.

FIG. 19B is a series of 3D dynamic MR lymphangiograms obtained 12 minutes post-injection of various non-PAMAM dendrimer based nano-size agents.

FIG. 20 is a chart showing the relative enhancement ratios of axillary lymph node-to-adjacent muscle for different Gd-based contrast agents.

FIGS. 21A-21C are a series of 3D dynamic MR images of solutions containing various amounts of G6 contrast agent under the same imaging conditions as the mouse studies shown in FIG. 19A and FIG. 19B. As shown, the T1-weighted MRI signal intensity increased with increasing concentration of G6 contrast agent. MRI of three sets of phantoms, high (6A, 0.1-40 mMGd; level 11000/window 22000), intermediate (6B, 0.1-1 mMGd; level 4000/window 8000) and low (6C, 0.01-0.1 mMGd; level 1200/window 2400) concentrations, are shown. G6 agent induces more MRI signal than Gd-DTPA for the same gadolinium concentration due to its macromolecular properties.

FIG. 22 is an axial image from a series of 3D dynamic MR lymphangiograms obtained at 24 minutes post-injection of the G6 nano-size contrast agents with phantom containing 400 and 800 ppm of Gd(III) of the G6 agent, demonstrating that high concentrations of Gd(III) can be achieved within the nodes.

FIG. 23 is a graph showing the change in gadolinium concentration over time in the left axillary lymph node of a subject mouse. Concentration was measured by setting the region of interest on the entire left axillary lymph node at the center slice. High concentrations in the lymph node are achieved by 12 minutes and are maintained to 60 minutes, with peaks at 24 and 36 minutes. The graph illustrates that the G6 agent is maintained in high concentration in the lymph node for up to 60 minutes post-injection.

FIG. 24 is a schematic diagram showing an exemplary magnetic resonance instrument for performing the disclosed methods.

### DETAILED DESCRIPTION

In order to facilitate review of the various embodiments of the invention, the following explanations of specific abbreviations and terms are provided:

LN—lymph node

SLN—sentinel lymph node

IL-15—interleukin-15

NK—natural killer

IEL—intraepithelial lymphocyte

CT—X-ray computed tomography

MR—magnetic resonance

MRI—magnetic resonance imaging

MRL—magnetic resonance lymphangiography

dmMRML—dynamic micro-magnetic resonance mammo-lymphangiography

2D-micro-MRL—two-dimensional micro-magnetic resonance lymphangiography

3D-micro-MRL—three-dimensional micro-magnetic resonance lymphangiography

2D-fastIR—two-dimensional fast-inversion recovery

SPGR—spoiled gradient echo

MIP—maximum intensity projection

USPIO—ultra-small particle of iron oxide

DAB—diaminobutane

DTPA—diethylenetriaminepentaacetic acid

GPDM—gadopentetate dimeglumine (Gd-DTPA-dimeglumine), a low molecular weight (0.94 kDa), FDA-approved extracellular MRI contrast agent also known as Magnevist™ (Schering AG, Berlin, Germany).

Gadomer-17—a low molecular weight (17 kDa) polylysine dendrimer-based magnetic resonance imaging agent (Schering AG, Berlin, Germany)

PAMAM—polyamidoamine

1B4M—2-(p-isothiocyanatobenzyl)-6-methyl-diethylenetriaminepentaacetic acid

DAB-G4D—generation-4 DAB-Am dendrimer

DAB-G5D—generation-S DAB-Am dendrimer

DAB-G6D—generation-6 DAB-Am dendrimer

DAB-G7D—generation-7 DAB-Am dendrimer

DAB-G8D—generation-8 DAB-Am dendrimer

PAMAM-G4D—generation-4 PAMAM dendrimer

PAMAM-G5D—generation-S PAMAM dendrimer

PAMAM-G6D—generation-6 PAMAM dendrimer

PAMAM-G7D—generation-7 PAMAM dendrimer

PAMAM-G8D—generation-8 PAMAM dendrimer

DAB-G4—DAB-Am-64-(Gd-1B4M)₆₄ dendrimer conjugate

DAB-G5—DAB-Am-128-(Gd-1B4M)₁₂₈ dendrimer conjugate

DAB-G6—DAB-Am-256-(Gd(1B4M)₂₅₆ dendrimer conjugate

DAB-G7—DAB-Am-512-(Gd-1B4M)₅₁₂ dendrimer conjugate

DAB-G8—DAB-AM-1024-(Gd-1B4M)₁₀₂₄ dendrimer conjugate

PAMAM-G4—PAMAM-G4D-(Gd-1B4M)₆₄ dendrimer conjugate

PAMAM-G5—PAMAM-G5D-(Gd-1B4M)₁₂₈ dendrimer conjugate

PAMAM-G6—PAMAM-G6D-(Gd-1B4M)₂₅₆ dendrimer conjugate

PAMAM-G7—PAMAM-G7D-(Gd-1B4M)₅₁₂ dendrimer conjugate

PAMAM-G8—PAMAM-G7D-(Gd-1B4M)₁₀₂₄ dendrimer conjugate

NCT—neutron capture therapy

NCA—neutron capture agent

NCE—neutron capture element

NaOH—sodium hydroxide

PBS—phosphate buffered saline

CCS—capture cross section

PT-18—a murine mast cell line

MIP—maximum intensity projection

Unless otherwise explained, all technical and scientific terms used herein have the same meaning as commonly understood by one of ordinary skill in the art to which this invention belongs. Definitions of common terms in magnetic resonance imaging may be found, for example, in Bushong, Magnetic Resonance Imaging: Physical and Biological Principles, Mosby, 1996. In the case of conflict, terms have the meanings provided in the present disclosure.

The singular terms “a,” “an,” and “the” include plural referents unless context clearly indicates otherwise. Similarly, the word “or” is intended to include “and” unless the context clearly indicates otherwise. The term “comprises” means “includes.” Molecular weights and formulas specifically recited are for illustrative purposes, and one of ordinary skill in the art will recognize that either may vary in practice from those specifically recited.

An “anti-tumor agent” is any agent (such as a radiological agent, a chemical compound or a biological entity) that has an anti-tumor effect, such as an anti-proliferative, cytotoxic or other anti-neoplastic effect. Anti-tumor agents need not have any specific level of activity or specific mechanism of operation, as long as they exhibit some therapeutic effect compared to a control. In certain implementations, anti-tumor agents reduce the size of a tumor by at least about 5%, such as at least 10%, 15%, 20%, 25%, or 30%.

Substances which may act as anti-tumor agents include a drug, a vaccine, a cytopathogenic substance, a neutron capture agent (NCA) for neutron capture therapy (NCT), a peptide, or an oligonucleotide. The anti-tumor agent is associated with a dendrimer conjugate, by any suitable means, such as by one or more of ionic bonding, covalent bonding, chelation, hydrogen bonding, van der Waals forces, metallic bonding, adsorption, encapsulation, or absorption.

Certain anti-tumor agents have a therapeutic effect when in the vicinity of tumor cells. Other anti-agents are taken up by tumor cells, or have a greater therapeutic effect when taken up by tumor cells. Certain anti-tumor agents require activiation, such as external activation, in order to exhibit a therapeutic effect.

Examples of anti-tumor agents are alkylating agents, antimetabolites, natural products, or hormones and their antagonists. Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide). Examples of hormones and antagonists include adrenocorticosteroids (such as prednisone), progestins (such as hydroxyprogesterone caproate, medroxyprogesterone acdtate, and magestrol acetate), estrogens (such as diethylstilbestrol and ethinyl estradiol), antiestrogens (such as tamoxifen), and androgens (such as testerone proprionate and fluoxymesterone). Examples of the most commonly used chemotherapy drugs that could be used in combination with the anti-IRX-5 agents includes Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol, Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. Examples of radioactive agents include radioactive isotopes, and an example of a biological anti-tumor agent is a monoclonal antibody that has an anti-tumor effect.

An “activatable anti-tumor agent” is an agent that is activated to exhibit a therapeutic or enhanced therapeutic effect. Activation may take place before or after administration of the agent. In at least certain examples, the anti-tumor agent is activated after it has accumulated or been transported to a treatment site. The activator may be located internal or external to a subject.

Activatable anti-tumor agents may be activated by a number of means, including by the application of physical energy, such as X-rays, microwaves, light, gamma rays, sound, ultrasound, neutrons, heat, antiproton therapy, protons, photon therapy, photodynamic therapy, electron beam therapy, pion therapy, or carbon ions. In further examples, the anti-tumor agent may be activated by the administration, or non-administration, of another substance, such as a chemical activator or inhibitor.

A “physical energy source” refers to a substance or device that may be used to supply physical energy, such as nuclear, radiative, thermal, or mechanical energy. Examples of physical energy include X-rays, microwaves, light, gamma rays, sound, ultrasound, neutrons, heat, antiproton therapy, protons, photon therapy, photodynamic therapy, electron beam therapy, pion therapy, or carbon ions. A physical energy source may be located internal or external to a subject to which physical energy is to be applied.

“Activatable by a neutron beam” refers to a substance that undergoes a physical or chemical change upon irradiation by a neutron beam. For example, a substance may absorb neutrons and undergo a radioactive process, such as fusion, fission, or nuclear rearrangement. A substance may break or form bonds when irradiated with a neutron beam. A substance may undergo structural chances, such as a change in conformation, upon neutron irradiation. In certain embodiments, the changes in a substance caused by neutron beam activation may be used for therapeutic effect, such as release or activation of a substance, such as a drug, or for radiation therapy, such as NCT.

The term “dendrimer conjugate” refers to a dendrimer attached or otherwise linked to another moiety, which may be a functional moiety, such as an imaging agent or an anti-tumor agent, such as a drug, a vaccine, a cytopathogenic substance, a neutron capture agent (NCA) for neutron capture therapy (NCT), a peptide, or an oligonucleotide. The moiety may be attached or linked to the dendrimer by any suitable means, such as by one or more of ionic bonding, covalent bonding, chelation, hydrogen bonding, van der Waals forces, metallic bonding, adsorption, encapsulation, or absorption. In certain examples, the dendrimer conjugate comprises a metal chelate. A number of moieties that may be included in dendrimer conjugates are discussed in U.S. Pat. No. 6,312,679.

Certain disclosed dendrimer conjugates are useful for imaging the lymphatic system of a subject (for example a mammal, such as a human or veterinary animal, including a horse, a cow, a sheep, a pig, a dog, or a mouse). Therefore, in one embodiment, a method for lymphatic system imaging is provided. The method includes administering an image enhancing amount of a dendrimer conjugate to a subject.

Any imaging technique, including MRI, CT, and lymphoscintigraphy may be used. However, MRI has several advantages over the other techniques for producing images of the lymphatic system. The spatial resolution of MRI (0.1-0.3 mm) is 30-100 times greater than that of scintigraphy (1 cm), and about 10 times greater than CT. Also, the temporal resolution of MRI is greater than 10 times that of scintigraphy, offering greater potential for dynamic studies of the lymphatics, for example, to identify sentinel lymph nodes based on a first appearance criterion. Moreover, three-dimensional images provided by MRI improve anatomical localization of imaged structures, and MRI does not involve exposure to radiation.

The disclosed dendrimer conjugates may also be useful in targeted anti-tumor therapy, such as to deliver an anti-tumor agent. In some examples, the anti-tumor agent is an activatable anti-tumor agent, such as anti-tumor agent that may be used in radiation therapy, such as NCT. For example, the dendrimer conjugates may be useful for performing NCT of the lymphatic system of a subject

The term “dendrimer” refers to a class of highly branched, often spherical, macromolecular polymers that exhibit greater monodispersity (i.e. a smaller range of molecular weights, sizes, and shapes) than linear polymers of similar size. These three-dimensional oligomeric structures are prepared by reiterative reaction sequences starting from a core molecule (such as diaminobutane or ethylenediamine) that has multiple reactive groups. When monomer units, also having multiple reactive groups, are reacted with the core, the number of reactive groups comprising the outer bounds of the dendrimer increases. Successive layers of monomer molecules may be added to the surface of the dendrimer, with the number of branches and reactive groups on the surface increasing geometrically each time a layer is added. The number of layers of monomer molecules in a dendrimer may be referred to as the “generation” of the dendrimer. The total number of reactive functional groups on a dendrimer's outer surface ultimately depends on the number of reactive groups possessed by the core, the number of reactive groups possessed by the monomers that are used to grow the dendrimer, and the generation of the dendrimer.

The term “metal chelate” refers to a complex of a metal ion and a group of atoms that serves to bind the metal ion (a “metal chelating group”). Typically, the metal chelating groups are attached to reactive groups on the surface (located, for example, at the termini of the dendritic branches) of the dendrimer. In some embodiments, a dendrimer conjugate may have fewer bound metal ions than it has metal chelating groups on its surface. For example, in particular embodiments at least 25%, 50%, 75%, 90%, or 95% of the metal chelating groups may have bound metal ions. Similarly, dendrimer conjugates may have fewer metal chelating groups than there are surface reactive groups on the dendrimer. For example, in particular embodiments at least 25%, 50%, 75%, 90%, or 95% of the surface groups of a dendrimer may be bonded to a metal chelating group. The differences in the number of metal chelates and the number of bound metal ions lead to the above-mentioned differences in chemical formulae and molecular weights.

The term “PAMAM dendrimer” refers to a dendrimer having polyamidoamine branches. As used herein, the term “DAB dendrimer” refers to a dendrimer having a diaminobutane core and polyalkylenimine branches. In general, DAB dendrimers may have polyalkylenimine branches, such as polyethyleneimine, polypropylenimine and polybutyleneimine branches. The term “DAB-Am dendrimer” refers to a DAB dendrimer having polypropylenimine branches and one or more surface amino groups, that is, amino groups at the ends of the last layer of branches that are added to the dendrimer, as it is grown from the initiator core, are terminated with one or more reactive amine groups. For example, when a DAB-Am dendrimer is synthesized using alkylenimine monomers, each successive layer of monomers that is added to the growing dendrimer to form additional branches provides a doubling of the number of free amine groups at the ends of the branches. The free amine groups at the ends of the branches (the surface of the dendrimer) may either be used as the reactive sites for adding an additional layer of monomers to the dendrimer to increase its generation or may be derivatized to provide alternative functional groups, such as quaternary amine groups or amide groups, on the surface of the dendrimer. Dendrimers of a particular generation and internal structure (core and branch structure), but with differing functional groups on their surfaces are commercially available.

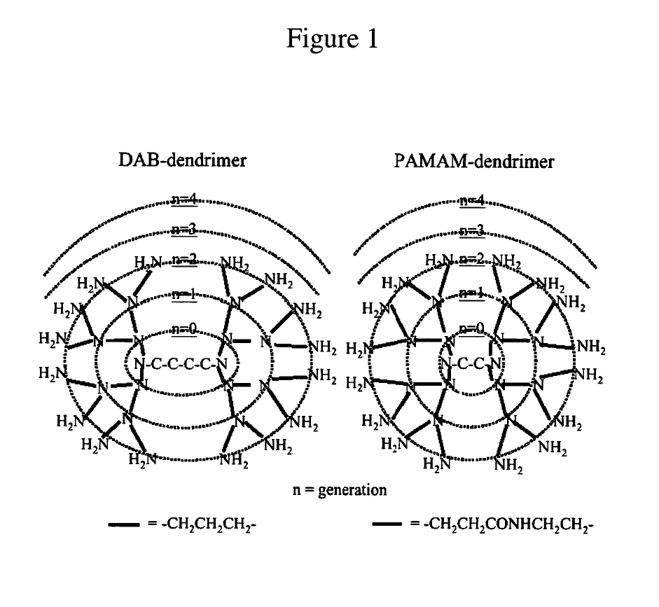

The term “DAB-Am-X” refers to a DAB-Am dendrimer having X number of surface amino groups. For example, DAB-Am-64 denotes a diaminobutane-core dendrimer having polypropylenimine branches and 64 amino groups at its surface. The structures of specific low-generation PAMAM dendrimers and low-generation DAB-Am dendrimers are compared in FIG. 1. FIG. 1 also illustrates the geometric increase in the number of branches and terminal amino groups with each successively higher generation of dendrimer. Of course, such amino groups appear as free (or surface) amino groups only at the ends of the branches. Otherwise in FIG. 1 internal amino groups are shown reacted with and bonded to additional branches that extend outward.

The term “bifunctional chelating agent” refers to a molecule that has at least two functional groups, one of which is a reactive group which can form a bond, such as a covalent bond, with another molecule, and one of which is a metal chelating group. Bifunctional chelating agents may be reacted with dendrimers to provide dendrimer conjugates, with metals added to the metal chelating group of the bifunctional chelating agent either before or after reaction of the bifunctional chelating agent with the dendrimer.

Conjugation between a dendrimer and another agent is a broad term that encompasses any joining together or coupling of the dendrimer with another agent, and this coupling can include formation of a covalent bond, ion-ion bonds, ion-dipole bonds, dipole-dipole bonds and hydrophobic interactions. In a particular example, the conjugate is formed by chelation of the agent to the dendrimer.

As used herein, the terms “administer” or “administering” refer to the addition of a substance to the body of a subject, for example local (as opposed to systemic) administration. In particular examples, the disclosed dendrimer conjugates may be administered by any appropriate route, including but not limited to intravenous injection, intralymphatic injection, parenteral injection, peritoneal injection, subcutaneous injection, intracutaneous injection, intratumoral injection, peritumoral injection, intradermal injection (such as into the areola), injection into the lymphatic system, injection into a surgical field, and subdermal injection. Other means of administration can be used, including oral, buccal, sublingual, and rectal administration and by intravenous or intraperitoneal infusion. NCAs may be prepared for administration by conventional pharmacological means, such as by adding excipients, fillers or diluents, buffers, stabilizers, flavorings, solubilizers, antibacterial agents, antifungal agents, isotonic agents, and the like.

In certain examples where lymphatic system components are to be imaged or treated with a dendrimer conjugate that includes an anti-tumor agent, such as in NCT, the site of intravenous, intralymphatic, parenteral, or subdermal injection is desirably, but not necessarily, in close proximity (such as less than 15 cm, 10 cm, or 5 cm away from) the lymphatic system components for which images are desired or to which the anti-tumor agent containing dendrimer conjugate is to be administered. However, in examples in which the lymphatic system is to be imaged and/or treated, the site and/or method of administration may be different, including administration at more remote locations.

An “image enhancing amount” refers to an amount that is sufficient to produce detectable (visually or electronically, such as by densitometry) differences in the image of lymphatic system components (such as lymph nodes and lymphatic vessels) relative to surrounding tissue at some time following administration of the dendrimer conjugate. For MRI, such differences may be detected in either a T₁- or T₂-weighted image taken at some time after the imaging agent is administered. The differences may be due to either an increase or a decrease in the intensity of the lymphatic system or a portion thereof, relative to surrounding tissue in comparison to an image obtained before administration of the agent. For example, the image intensity of one or more components of the lymphatic system will be increased (or decreased) in intensity relative to surrounding tissue by greater than about 20%, 50%, 75%, or 90% when compared to an image obtained without administering or to regions of an image that are not enhanced by the contrast agent. Other anatomical structures may or may not exhibit enhancement following administration of the dendrimer conjugate.

Differences in signal intensity between the lymphatic system, parts of the lymphatic system, and the surrounding tissue may be used to detect and/or differentiate one or more conditions of the lymphatic system, such as the location of particular components of the lymphatic system (including lymphatic vessels and lymph nodes), the presence of metastatic cells in lymph nodes, swelling of lymph nodes, and dilation of lymphatic vessels. In general, components of the lymphatic system will have a positive contrast (increase in image intensity) in a T₁-weighted image relative to surrounding tissue, especially where the dendrimer conjugate is a T₁ agent. For example, where a T₁-weighted image is obtained following administration of an image enhancing amount of a disclosed dendrimer conjugate that includes gadolinium ions (which increase the longitudinal relaxation rate 1/T₁ more than the transverse relaxation rate 1/T₂), lymphatic system components will appear brighter in a T₁-weighted image than surrounding tissue.

The increase in image intensity of the lymphatic system relative to surrounding tissue permits localization of the lymphatic system components in such an image. Furthermore, where a lymph node contains metastatic tumor cells, the afferent lymphatic vessel leading to the metastatic lymph node may not only appear brighter than surrounding tissue, but also larger (dilated) than afferent lymphatic vessels leading into normal lymph nodes. In addition, swollen lymph nodes that contain metastatic tumor cells may be observed to have a bright fringe and a dark center, indicative of infiltration of the metastatic tumor cells that block entrance of the dendrimer conjugates into the germinal center of the lymph node.

In contrast, swollen lymph nodes caused by infection do not exhibit a lack of contrast in the center. In addition, bright images of lymphatic vessels associated with infected and swollen lymph nodes (larger by comparison to non-infected lymph nodes seen elsewhere in a particular subject or larger by comparison to lymph nodes typically seen in normal patients) may also appear irregular, and aid in identifying swelling associated with infection rather than the presence of metastatic cancer cells. Thus, differences in the image intensities associated with the different parts of an enhanced image of a lymphatic structure (relative to surrounding tissue) can be used to identify and/or differentiate conditions of the lymphatic system.

Conversely to a T₁-image, lymphatic system components will generally have negative contrast (appear darker) in a T₂-image relative to surrounding tissue. For example, where the dendrimer conjugate includes iron ions (a T₂-agent), dark lymphatic vessels and lymph nodes will appear against a bright background of surrounding tissue. Metastatic lymph nodes will appear with a dark fringe and a bright center in a T₂-weighted image, and the dark areas indicative of afferent vessels may appear dilated. Swollen lymph nodes and dilated lymphatic vessels (such as induced by infection) will appear as larger dark areas when compared to typical corresponding normal lymph nodes and non-dilated vessels.

In particular embodiments, administering an imaging enhancing or therapeutic (for example, anti-tumor) amount of the dendrimer conjugate includes administering a dose between about 0.0001 mmol metal/kg of the subject's body weight and about 1.0 mmol metal/kg of the subject's body weight, for example, between about 0.001 mmol metal/kg and about 1.0 mmol metal/kg, such as between about 0.01 mmol metal/kg and about 1.0 mmol metal/kg. In other particular embodiments, image enhancing or therapeutic (for example anti-tumor) amounts of the dendrimer conjugates are provided by administering the dendrimer conjugates in dosages that are 1/50^{th} to ⅓ of the molar dosages on a dendrimer basis or 1/2500 to 1/500 of the molar dosage on a metal ion basis (such as gadolinium ion basis) as required for simple chelates such as Gd-DOTA and Gd-DPTA (which are typically administered in a range of 0.1 to 1.0 mmol Gd/kg). In other particular embodiments, a detectable difference in lymphatic system MRI image intensity may be provided by administering between about 0.0001 mmol Gd/kg and about 1.0 mmol Gd/kg, for example, administering between about 0.01 mmol Gd/kg and about 1 mmol Gd/kg, such as administering between about 0.1 mmol Gd/kg and about 1 mmol Gd/kg intravenously, parenterally, intratumorally, peritumorally, intradermally (such as into the areola), subdermally, or into a surgical field.

Imaging may begin immediately or anywhere from about 1 minute to about 120 hours after administration, such as between about 3 minutes and about 24 hours after administration, or between about 3 minutes and about 60 minutes after administration. The time before imaging may be altered based on the particular dendrimer conjugate used and its physiological properties, such as the time it takes to accumulate in an area of interest and its retention time.

Imaging, once begun, may be continued for any subsequent amount of time that facilitates analysis of the images for a particular purpose (for example, to follow flow of the lymph fluid). For example, if identification of a sentinel lymph node is desired, a single image that is obtained anywhere between 2 and 60 minutes following administration by intratumoral administration may be sufficient. On the other hand, a series of images obtained at various points in time from administration to a desired time after administration, such as several hours or more, may be obtained if lymphatic flow beyond the sentinel lymph node is to be imaged or if intraoperative (during a surgical procedure) or intratreatment (such as during administration of a dendrimer conjugate which includes an anti-tumor agent, such as during NCT) localization of one or more particular lymph nodes is desired. Obtaining images at various times after administration may aid a surgeon performing a partial or full lymphadenectomy or in the administration of NCT.

For example, a series of images may be obtained successively over a period of time where each image is separated by any amount of time from the instrumental limit for successive image acquisitions to minutes or hours apart, such as 5, 10, 15, 30 minutes apart or 1, 2 or 3 hours apart. Imaging may be done before or during surgery or therapy, and continued for any period during surgery or therapy, for example, to help a surgeon guide a needle to a lymph node for a biopsy or to position an activator for an activatable anti-tumor agent, such as a radiation source for NCT. Since surgical instruments will appear brighter than surrounding tissue in a T₁-weighted image, it is desirable to use a series of T₁-weighted images in conjunction with administration of a T₁-agent, such as a dendrimer conjugate including gadolinium ions, to permit simultaneous visualization and localization of the surgical instrument and the lymphatic system component(s) on which the surgeon will act with the instrument. Surgical instruments may also be used to aid in positioning an activator for an activatable anti-tumor agent, such as a radiation source for NCT.

“Neutron capture element” (NCE) refers to an atom which absorbs neutrons and, after doing so, produces products such as radiation energy, particles, and elements, which may be used therapeutically, such as in NCT, to treat diseased cells, such as metastatic cells or neoplastic tissue. A NCE preferably has a large neutron capture cross section and may be a single isotope or a mixture of isotopes, including a sample of an element containing a distribution, such as a natural distribution or a sample enriched in one or more isotopes, of isotopes, at least one of which is suitable for use in NCT. Examples of NCEs include boron and gadolinium, more particularly, ¹⁰B, ¹⁵⁵Gd, and ¹⁵⁷Gd. However, other elements and isotopes may be used. Suitable NCEs have a larger neutron capture cross section than the elements making up surrounding tissue, preferably larger than about 100 barn, more preferably larger than about 5000 barn.

Although the neutron capture products of ¹⁰B are believed to be more destructive to malignant cells than the products of ¹⁵⁷Gd, gadolinium has a number of advantages over ¹⁰B in NCT. For example, gadolinium complexes are often easier to introduce into the environment, or inside, of malignant cells. The gamma radiation produced when gadolinium isotopes absorb a neutron has a longer range than the alpha particles emitted from ¹⁰B.

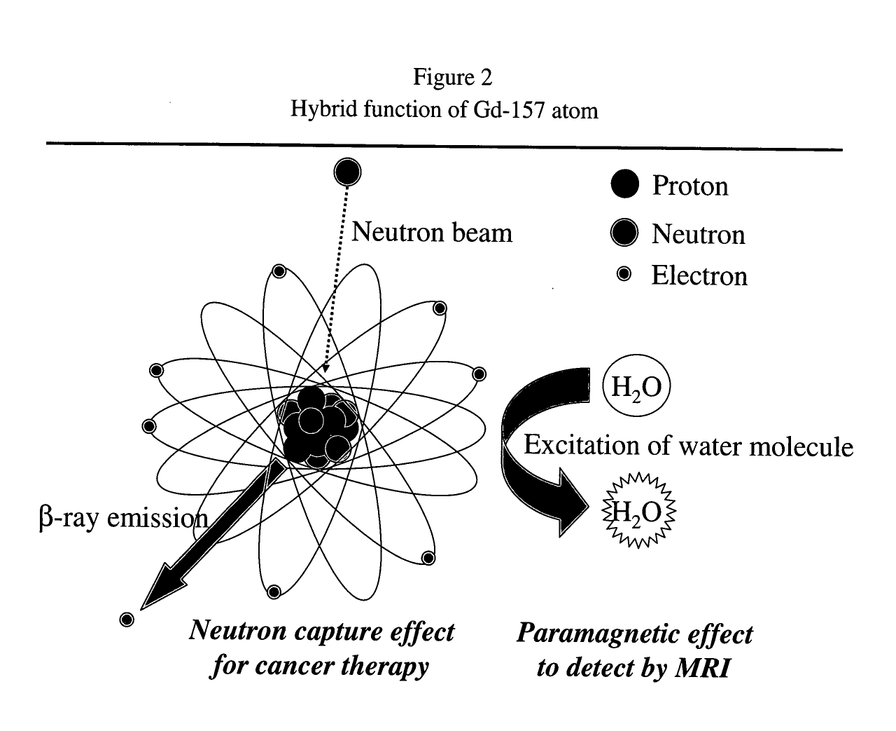

In addition, gadolinium complexes are known contrast agents for imaging techniques, including MRI, and all gadolinium isotopes, both stable and radioactive, can serve as MR contrast agents due to their paramagnetic characteristics. Therefore, gadolinium complexes may be created that serve both as contrast agents and as therapeutic agents. A dual contrast agent/neutron capture agent (NCA) can allow radiation timing to be optimized during NCT by observing when the desired concentration of NCA is in the area to be treated. Furthermore, the existence of clinically used gadolinium contrast agents also means that the pharmacokinetic and pharmacological properties of gadolinium compounds are well studied, which may help in designing or selecting a NCA for a particular use. The dual roles of contrast agent and NCE that may be played by gadolinium complexes are illustrated in FIG. 2.

“Neutron capture agent” (NCA) refers to a NCE and a molecule to which the NCE is complexed, bound, bonded, or otherwise associated with, such as by one or more of ionic bonding, covalent bonding, chelation, hydrogen bonding, van der Waals forces, metallic bonding, adsorption, encapsulation, or absorption. NCAs are preferably non-toxic and excreteable from the body, particularly if multiple treatments are to be administered. Water solubility may be desirable for NCAs in order to reduce or avoid the need for a co-solvent and to potentially reduce the volume of material needed to be administered to a subject in order to deliver the desired amount of NCA. For example, the water solubility may be at least about 0.1 mg/mL, more preferably at least about 100 mg/mL.

It may be desirable that the NCA have an affinity or specificity for tissue or cells to be treated or for a particular area of the body. It may also be desirable that the NCA be capable of reaching sufficient concentrations and of being retained in the treatment site long enough for treatment to be administered. Moreover, because of the limited range of the radiation absorption products produced by NCEs, such as the Auger electrons which result in much of the therapeutic potential of gadolinium, the NCA may be more effective if it can be taken up by a cell, rather than being in the surrounding extracellular environment. In particular, it would be beneficial if the NCA is taken up by the nucleus, rather than remaining in the cytoplasm.

Typically, each NCA is associated with on or more NCEs. In some aspects, each NCA is associated with a plurality of NCEs. NCAs having multiple NCEs may increase the amount of therapeutic radiation produced by each NCA molecule. Using such a NCA may allow the dosage, length of treatment, or level of radiation to be reduced.

In some aspects, the NCA can be monitored in vivo. For example, NCAs which incorporate gadolinium may be detected by imaging techniques such as MRI. However, other tags, such as optical, radioactive or fluorescent tags, may be added to the NCA. For example, the radioactive isotope ¹⁵³Gd may be used as both a radioactive tracer and as a contrast agent.

In certain implementations, the NCA includes a dendrimer. For example, the NCA may include a dendrimer conjugate of a dendrimer and a metal chelate, where the metal is a NCE. The dendrimer may be a PAMAM dendrimer, a DAB-Am-X dendrimer, a polylysine dendrimer, or other dendrimer.

Different dendrimer conjugates behave differently in vivo. In particular, the pharmacological or pharmacokinetic properties of the dendrimer conjugates may be related to the size of the dendrimer used in the dendrimer conjugate. Accordingly, the NCT specific application, such as tissue or cells to be imaged or treated via NCT, may influence the selection of a particular dendrimer conjugate, or mixture thereof, to be administered.

For example, when the target area for treatment is the lymphatic system, it may be preferable to use a NCA that is small enough to enter lymphatic vessels, yet large enough to be retained within the lymphatics and not leak from the capillary vessels. In at least one implementation, suitable NCAs for the lymphatic system are preferably larger than about 4 nm in diameter, such as dendrimer conjugates having a diameter of at least about 4 nm. Smaller molecules may diffuse into surrounding tissue, possibly resulting in poor signal to background ratios if the NCA is also an imaging or contrast agent. The diffusion of smaller molecules may also reduce the amount of NCA available for NCT.

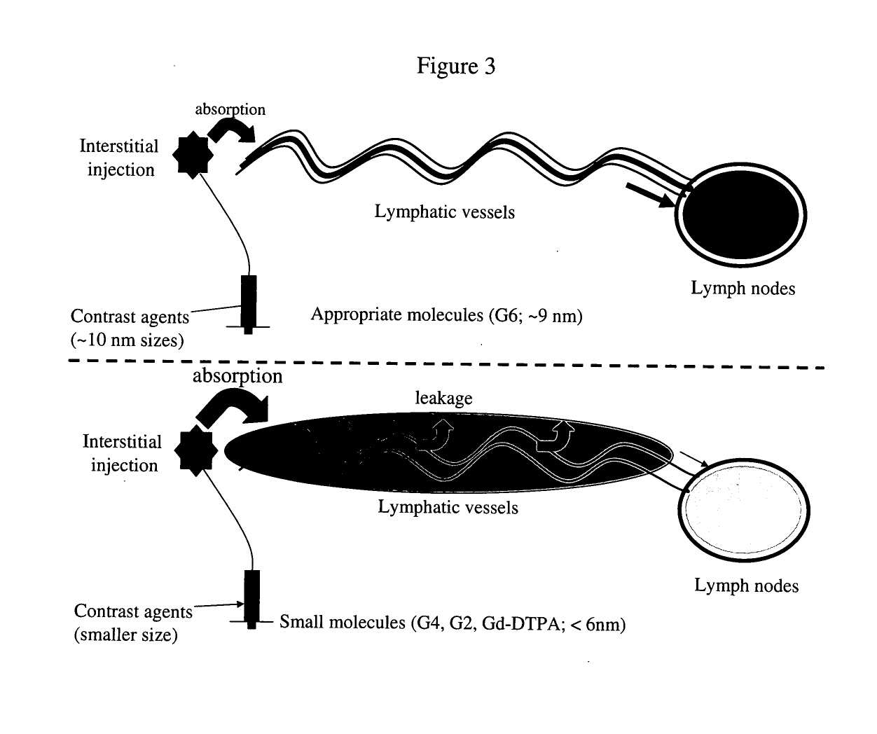

The NCA is preferably smaller than about 12 nm. Molecules larger than about 12 nm may more slowly diffuse from interstitial space and accumulate more slowly in lymph nodes, including sentinel nodes, thus potentially resulting in longer wait times before the nodes can be treated or imaged. One suitable NCA, as well as a suitable imaging or contrast agent, includes a PAMAM-G6 dendrimer, preferably a gadolinium PAMAM-G6 dendrimer conjugate. The gadolinium PAMAM-G6 dendrimer conjugate is retained in the lymphatic vessels and has an affinity for normal lymph nodes. Other NCAs, such as the Gadomer-17 dendrimer conjugate (which is also an imaging or contrast agent), may be used. The effect of NCA size on the behavior of the NCA in the lymphatic system is illustrated in FIG. 3.

The physical and chemical properties of the NCA, such as its ability to be taken up by cells or its hydrophilicity, may also be tailored to a specific imaging or treatment use. For example, hydrophobic substances may be more rapidly cleared from circulation by the liver and kidneys. In another implementation, NCAs may be chemically modified to have a greater affinity for, or retention in, a particular cell type, tissue type, or treatment area. For example, the NCA may be associated with an antibody which targets a particular type of cell, such as a tumor cell.

“Neutron source” refers to a source of slow (thermal) or fast neutrons which may be used in NCT. Fast neutrons generally have a kinetic energy of about 1 MeV. Fast neutrons may be generated by any suitable source, including cyclotrons. Fast neutron sources include those that are commonly used in conventional radiotherapy. Fast neutron sources may also be used to produce slow neutrons, such as by passing a fast neutron beam through a moderator, such as heavy water, light water, or graphite. Dosages of fast neutrons used in NCT are generally less than are used in conventional radiotherapy due to the therapeutic enhancement provided by the NCA.

Slow neutrons, sometimes called thermal neutrons, may be obtained from any suitable source. Slow neutrons typically have a kinetic energy of about 0.025 eV, similar to the average kinetic energy of room temperature molecules. Because of their lower energy, slow neutrons may be less penetrating than fast neutrons, but are typically less damaging to tissue. Slow neutron sources may also be used to produce epithermal neutrons, which typically have energies of about 0.5 eV to about 10,000 eV and may also be used for NCT.

As previously mentioned, fast neutron beams may be passed through a moderator in order to produce a suitable beam of slow neutrons or epithermal neutrons. Examples of neutron sources that may produce neutrons ranging from about 0.025 eV to about 10 MeV are described in U.S. Pat. No. 5,976,066, and are typically obtained by moderating a more energetic neutron beam. Fast neutron sources, and their moderation into slow neutrons, are also discussed in U.S. Pat. No. 6,770,030. Described neutron sources included moderated fast neutron beams produced by fission of ²³⁵U, spontaneous fission of ²⁵²Cf (typically producing neutrons having an average kinetic energy of 2.3 MeV), and mixtures of particle emitting isotopes, such as mixtures of ²³⁹Pu or ²²⁶Ra, with ⁹Be. In addition, the use of deuterium/tritium accelerators is known to produce neutrons when generated particles collide with a metal hydride target.

While the neutron source is not critical, there are a number of properties which may be desirable in a neutron source. The neutron beam is preferably free of other radioactive components, such as gamma rays, beta radiation, and X-rays, which may cause undesirable side effects. The neutron source preferably is capable of producing a steady, controllable neutron stream. It may be beneficial if the neutron source can be pulsed, and turned on and off, to increase its ease of use and storage. A neutron source that is constantly on may require shielding when the neutron source is not being used. Similarly, the neutron source preferably may be focused in a beam, more preferably a beam having an alterable size and position, so that the beam may be focused to a particular size and on a particular area to which treatment will be administered.

The average flux of neutrons through the subject tissue may be varied as desired. In at least certain examples, flux may range from about 1 n/cm² to about 1×10¹⁴ n/cm², preferably between about 1×10⁸ n/cm² to about 10×10¹² n/cm². The average kinetic energy of the neutrons may range from about 0.001 eV to about 10 MeV. When slow NCT is used, the average kinetic energy of the neutrons used is more preferably from about 0.02 eV to about 10,000 eV.

“Treatment time” refers to the duration for which an activator for an activatable anti-tumor agent, such as neutron beam from a neutron source, is applied to a subject. The treatment time may vary, including based on the anti-tumor agent or activator used (for example, in NCT, whether natural or enriched gadolinium is used), the size or location of the treatment area, or the susceptibility of the target cells to the anti-tumor agent. When the activator is a neutron source, the treatment time may be affected by the neutron flux used or the average energy of the neutrons. In at least one aspect, treatment times range from about 1 minute to about 3 hours, more preferably from about 30 minutes to about 70 minutes. Treatments may be administered multiple times over a given time period, if desired. If multiple treatments are administered over time, the duration of each treatment may be the same or may vary.

A “effective amount” of an anti-tumor agent, such as an NCA, refers to an amount of anti-tumor agent that, after administration to a subject, is sufficient to cause damage to target cells when the anti-tumor agent is proximate the target cells and, if the anti-tumor agent is an activatable anti-tumor agent, is subjected to an activator (such as, for NCT, a beam of neutrons from a neutron source). The amount of anti-tumor agent needed to be administered in order to be therapeutically effective may depend on a number of factors, including the nature or location of target cells, the method of administration, the form of the anti-tumor agent, or the activator (such as the particular radiation source used).

For example, different anti-tumor agents are processed differently by various tissues, cells, or treatment areas. If the target tissue, cells, or treatment area has a high affinity for the anti-tumor agent, a lower dose may be needed. Similarly, if the anti-tumor agent is taken up inside the target cells, more preferably by the cell nucleus, lower anti-tumor agent doses may be required than if the anti-tumor agent remains in the extracellular environment. Other factors which may affect dosage include the subject's age, weight, sex, general health, and prior medical history.

In at least certain examples, it is desirable to have at least one anti-tumor agent proximate each target cell. However, depending on the accessibility of the target cells, multiple administrations of the anti-tumor agent, and activator treatments, if needed, may be given in order to expose all target cells to the anti-tumor agent. In the case of NCT, certain NCAs are associated with a plurality of NCEs. Generally, the more NCEs that are associated with each NCA, the lower overall the dosage of NCA that will be required.

In some examples, cellular concentrations of anti-tumor agents needed to be therapeutically effective, including for certain gadolinium dendrimer conjugates, are between about 0.01 μg/mL to about 10,000 μg/mL, more preferably about 100 μg/mL to about 6,000 μg/mL. In additional aspects, the dosage for a therapeutically effective treatment is about 0.1 mg/kg to about 500 mg/kg of subject body weight. In yet additional examples, suitable treatment dosages range from about 0.1 μg to about 50 g, such as from about 0.5 μg to about 50 μg, or from about 0.1 mg about 2 g, per treatment. In terms of molar concentrations, therapeutically effective amounts may be from about 0.1 μM to about 100 mM, for example from about 0.1 μM to about 40 mM. In terms of parts per million, therapeutically effective amounts may be from about 10 ppm to about 10,000 ppm, such as from about 200 ppm to about 7,000 ppm. In certain examples, when the anti-tumor agent is a gadolinium containing NCA, the cellular concentration of Gd(III) is between about 100 ppm and about 2000 ppm, such as between about 400 ppm and about 1000 ppm. The therapeutically effective amount of anti-tumor agent may also be an image enhancing amount of anti-tumor agent, and vice versa.

“Selectively concentrating” refers to introducing a substance, such as a dendrimer conjugate, preferentially to a particular area, such as an area of a subject to be treated. For example, the substance may be preferentially introduced to a particular physiological area or system of the body, a particular tissue, or a particular type of cells, generally referred to as the “target area.” For example, a substance may be selectively concentrated in the lymphatic system of a subject, such as in the lymphatic vessels and/or lymph nodes. Selective concentration in the lymphatic system refers to a concentration that is greater in the lymphatic system than in other tissue to a sufficient extent to provide a diagnostic or therapeutic advantage, such as the ability to image or provide therapy to the target.

Substances may be selectively concentrated in the target area by any suitable method. For example, the chemical, physical, physiological, or pharmacokinetic properties of a substance may be chosen such that the substance is preferentially introduced to, or retained by, the target area, as opposed to other areas of the subject. For example, the size of a substance may affect its ability to be introduced or retained by a particular target area. Similarly, the hydrophilicity of a substance may affect its ability to be concentrated in a target area. In certain implementations, the substance may be modified to increase the substance's affinity for the target area, such as by modifying the substance to increase its uptake by target cells.

“Neutron capture therapy” (NCT), refers to methods for treating diseased or damaged tissue or cells by administering a NCA to a subject and then irradiating the subject with neutrons, for example a beam of neutrons from a neutron source, such that at least a portion of the radiation is absorbed by the NCA. When the NCA absorbs neutrons, it emits particles or other radiation products which may be therapeutically used to damage or kill target cells or tissues, such as neoplastic cells or tissue or metastatic cells. Methods for NCT are discussed in PCT Publication WO 96/00113, U.S. Pat. Nos. 6,770,020 and 5,976,066 and by De Stasio et al., Cancer Res.; 61:4272, 2001; Tokumitsu et al., Cancer Let.; 150:177, 2000; and Hofmann et al., Invest. Radio.; 34(2): 126, 1999.

NCT may begin immediately or anywhere from about 1 minute to about 120 hours after administration of the NCA, such as between about 3 minutes and about 24 hours after administration, or between about 3 minutes and about 60 minutes after administration. The time before NCT may be altered based on the particular NCA, such as a particular dendrimer conjugate used, and its physiological properties, such as the time it takes to accumulate in an area of interest and its retention time. NCT, once begun, may be applied for a particular treatment time.

In particular embodiments, the anti-tumor agent, which may also optionally be an imaging or contrast agent, is a dendrimer conjugate, such as a dendrimer conjugate comprising a DAB-5, generation 2 polylysine, PAMAM-G6D, PAMAM-G7D, or PAMAM-G8D dendrimer and a metal chelate. In certain examples, regardless of the dendrimer conjugate used, a difference in an image signal intensity of at least a portion of the lymphatic system that appears after the dendrimer conjugate is administered is used to image the components of the lymphatic system, including lymphatic vessels and lymph nodes. A particular component of the lymphatic system may be exposed to an activator, such as neutron beam from a neutron source, based on the images obtained in order to activate an activatable anti-tumor agent.

In more particular embodiments, the dendrimer of the dendrimer conjugate is DAB-G5D, PAMAM-G6D or PAMAM-G7D, and in more particular embodiments the dendrimer is PAMAM-G6D. These dendrimers, and the ones mentioned before, may be conjugated to a variety of moieties, including metal chelates. Specific examples of metal chelates that may be used include DTPA metal chelates, DOTA metal chelates, DO3A metal chelates, DOXA metal chelates, NOTA metal chelates, TETA metal chelates, DOTA-N(2-aminoethyl)amide metal chelates, DOTA-N-(2-aminophenethyl)amide metal chelates, BOPTA metal chelates, HP-DO3A metal chelates, DO3MA metal chelates, or 1B4M metal chelates. The element of the chelate may be a NCE, preferably one or more isotopes of gadolinium(III).

In other more particular embodiments, the metal chelate of the dendrimer conjugate is a 1B4M metal chelate of gadolinium (III) ions and the dendrimer conjugate is DAB-G5, Gadomer-17, PAMAM-G6, PAMAM-G7 or PAMAM-G8. More particularly, the dendrimer conjugate may be DAB-G5, PAMAM-G6, or PAMAM-G7. In specific embodiments, the dendrimer conjugate is PAMAM-G6.

Any of the anti-tumor agents, including dendrimer conjugates, that are disclosed may further comprise an optical, radioactive, or fluorescent moiety to aid in location of lymphatic system components during a surgical procedure or medical treatment. As used herein the terms “optical moiety” and “fluorescent moiety” refer to a moiety that may be visualized by the naked eye or a photon detector (for example, a charge-coupled device) by virtue of its absorption or emission of visible light, respectively. Examples of optical and fluorescent moieties include, respectively, an isosulfan blue dye or a fluorescent molecule. In the case of a fluorescent moiety, visualization of the moiety may include illumination of the moiety with ultraviolet light to stimulate emission of fluorescent photons. As used herein, the term “radioactive” refers to a moiety that emits radiation, such as may be detected by a radiation detector, such as a scintillation counter.

Specific components of the lymphatic system that may be imaged, or treated by an anti-tumor agent, such as a NCA, include lymph nodes and lymphatic vessels, regardless of their location in the subject's body. In particular embodiments, a DAB-G5, Gadomer-17, or PAMAM-G4 dendrimer conjugate is used to image or treat lymph nodes, or a PAMAM-G8 or Gadomer-17 dendrimer conjugate is used to image or treat lymphatic vessels. In other particular embodiments, a PAMAM-G6 dendrimer conjugate is used to image or treat the lymphatic system, including the lymphatic vessels and the lymph nodes.

Also disclosed is a method for identifying a lymph node into which lymph fluid flows from a tumor, such as a breast tumor. This particular method includes administering an image-enhancing amount of a dendrimer conjugate to an intratumoral, peritumoral, intradermal (such as the areola) site of administration. A path of lymph fluid flow from the site of administration is imaged using magnetic resonance imaging to provide an image of the lymphatic system surrounding the tumor. From this image, the lymph node may be identified along the path of lymph fluid flow from the site of administration. The method also may include detecting metastatic tumor cells in the node by detecting an image filling defect of at least a portion of the sentinel node. Alternatively, the path of lymphatic flow is imaged over time (such as for periods as described above and below) to observe a lymph node that is first in time to receive the dendrimer conjugate following administration of the dendrimer conjugate to the site of administration near or in the tumor. In some embodiments, the dendrimer conjugate used for this method is DAB-G4, DAB-G5, DAB-G6, DAB-G7, DAB-G8, PAMAM-G4, PAMAM-G5, PAMAM-G6, PAMAM-G7, PAMAM-G8, or Gadomer-17. In particular embodiments, the dendrimer conjugate is DAB-G5, PAMAM-G6, PAMAM-G7, PAMAM-G8, or Gadomer-17, and in specific embodiments, the dendrimer conjugate is PAMAM-G6. As before, the dendrimer conjugate may also include an optical, radioactive, or fluorescent moiety.

Once the lymph node, lymphatic vessel, or metastatic cells of interest are located, the area of interest may be treated with an activator to activate an activatable anti-tumor agent, such as for NCT. For example, treatment may involve exposing an area of a subject to a beam of neutrons from a neutron source. Optical, radioactive, or fluorescent moieties conjugated to the dendrimer conjugate may further assist localization of lymphatic system components during NCT to treat lymphatic system components. In certain implementations, the imaging agent is a NCA.

Also disclosed are methods for performing NCT. In certain implementations, the method includes administering a therapeutically effective amount of a dendrimer conjugate, such as DAB-5, PAMAM-G6, PAMAM-G7, PAMAM-G8, or Gadomer-17. In a specific example the dendrimer conjugate is PAMAM-G6.

After the NCT is administered, the treatment area may be exposed to a radiation source, such as a neutron beam from a neutron source, for a treatment time. If desired, fluorescent, optical, or radioactive tags may be used to assist in identifying the area to be treated. In certain implementations, the therapeutically effective amount of the NCA is also an image enhancing amount and the NCA may be used to both locate the treatment area and in treating the area. In certain examples, the treatment area is a component of the lymphatic system.

In addition to NCT, the dendrimers or dendrimer conjugates, including PAMAM-G6 are used as a delivery vector for other means of therapy, such as those discussed in U.S. Pat. No. 6,312,679. For instance, delivery of anti-cancer drugs, cytopathogenic substances, or anti-sense oligo-DNA or siRNA into lymph nodes is accomplished using a dendrimer or dendrimer conjugate. For example, the encapsulation of the anticancer drugs adriamycin and methotrexate by PAMAM dendrimers is discussed in Kojima et al., Bioconj. Chem.; 11:910-917, 2000. Covalent bonding of a dendrimer to the anticancer drug doxorubicin is discussed in Padilla De Jesus et al., Bioconj. Chem.; 13:453-461, 2002.

The dendrimer is conjugated to an anti-tumor agent that is selectively concentrated in the lymphatic system, which is particularly helpful for the treatment of metastases or micro-metastases. In particular examples, the dendrimer conjugate may be activated once it has concentrated in a target location, such as in a lymph node where metastatic cells are or may be present, including the methods described in PCT Publication WO 2004/009135. Activatable anti-tumor agents may be activated by X-rays, microwaves, sound, light, heat, gamma rays, ultrasound, neutrons, antiproton therapy, protons, photon therapy, photodynamic therapy, electron beam therapy, pion therapy, or carbon ions. For example, activation can take the form of a neutron beam that induces the release of anti-tumor agents, such as radioactive or high energy particles or a cytotoxic agent. A less hydrophilic dendrimer that more selectively persists in the lymph node is an example of a conjugate that is particularly effective for such a use.

The dual nature of the disclosed dendrimer conjugates as imaging and therapeutic agents provides an advantageous combination in which the agent can be administered for selective targeting to the lymphatic system, where the conjugate concentrates. One or more lymph nodes (such as a sentinel node) can then be effectively and efficiently imaged using the conjugate's properties as a contrast agent, and the imaging is then used to target an externally applied activator, such as a beam of energy (for example a neutron beam) to the anatomic site where the concentration of the contrast agent has been located. In this manner, the external energy is applied selectively to a target site, thereby substantially sparing nearby non-target tissue from injury.

Various embodiments are specifically illustrated by the following examples.

### EXAMPLE 1

### Preparation and Administration of a Dendrimer Conjugate to Detect and Localize a Lymph Node

This example describes MRI imaging of the lymphatic system of mice by using a PAMAM-G6D dendrimer conjugate, specifically PAMAM-G6, which is a Gd-1B4M conjugate. Imaging of lymphatic drainage associated with breast tumors using PAMAM-G6 is shown to provide sufficient temporal and spatial resolution to accurately identify and locate lymph nodes. Sentinel lymph nodes may also be identified based on a first appearance criterion using time series of images. Image-based assessment of the disease state of the components of the lymphatic system is also demonstrated.

### I. Preparation of the Contrast Agent

The generation-6 polyamidoamine (PAMAM-G6) dendrimer (Dendritech, Inc., Midland, Mich.) has an ethylenediamine core, 256 terminal reactive amino groups, and a molecular weight of 58,048 Da. The PAMAM-G6D dendrimer was concentrated to about 5 mg/ml and diafiltrated against 0.1 M phosphate buffer at pH 9. The PAMAM-G6D dendrimer was reacted with a 256-fold molar excess of 2-(p-isothiocyanatobenzyl)-6-methyl-diethylenetriamine-pentaacetic acid (1B4M) at 40° C., and maintained at pH 9 with 1 M NaOH for 24 hours. An additional equal amount of the 1B4M was added after 24 hours as a solid. The resulting preparations were purified by diafiltration using a Centricon 30 (Amicon Co., Beverly, Mass.). This resulted in over 98% of the amine groups on the dendrimer reacting with the 1B4M as determined by ¹⁵³Gd (NEN DuPont, Boston, Mass.) labeling of aliquots, as described in Kobayashi, et al., Mag. Res. Med.; 45: 454-60 2001.

Subsequently, PAMAM-G6 dendrimer-1B4M conjugate (about 3 mg containing 4 μmol 1B4M) was mixed with 8 μmol of non-radioactive Gd(III) citrate in 0.3 M citrate buffer overnight at 40° C. The excess Gd(III) in the preparation was removed by diafiltration using a Centricon 30 filter (Amicon Co.) while simultaneously changing the buffer to 0.05 M PBS. The purified sample was diluted to 0.2 mL with 0.05 M PBS and about 5 μL was used in each mouse breast tissue. A replacement assay using ¹⁵³Gd was used to determine that 84% of the 1B4M on the PAMAM-G6 dendrimer-1B4M conjugate was indeed chelating Gd(III) atoms, as described by Kobayashi, et al., which reference is cited in the preceding paragraph.

GPDM (Magnevist™ with a molecular weight of 938 Da), an FDA-approved extracellular MRI contrast agent (Schering AG, Berlin, Germany), was used as a control.

### II. Preparation of the Mice

Normal and breast-tumor bearing mice were prepared. Twelve week-old Balb/c mice (n=5) or athymic nu/nu mice (n=9) (NCI, Frederick, Md.) were used as the normal mice.

The breast-tumor bearing mice were thirty one week-old BALB-neuT mice (n=3) transgenic for the rat HER-2/neu (Erb B2) oncogene under the control of the mouse mammary tumor virus promotor (MMTV). These mice exhibit tissue-specific expression of HER-2/neu. BALB-neuT mice were used because of their spontaneous development of bilateral breast cancers and lymph node metastases. Heterozygous female BALB-neuT mice (BALB/c background) develop mammary gland lobule hyperplasia at 5-6 weeks of age that progresses to atypical hyperplasia by 8-9 weeks, followed by in situ carcinoma by 14 weeks, becoming invasive carcinoma usually by 21 week of age. Rovero et al., J. Immunol.; 165:5133-42, 2000. Most metastatic lymph nodes from the mouse mammary pad localize in the neck, lateral thoracic, or axillary region.

Tumor xenografts of PT-18, a murine mast cell line, were introduced into the left mammary pad in athymic nu/nu mice. When 10⁷ PT-18 cells were injected in the left mammary pad of 10 athymic nu/nu mice, six mice developed tumor masses in the left axillary lymph nodes within three weeks.

### III. Adminstration of the Contrast Agent and Imaging

Mice were anesthetized with 1.15 mg sodium pentobarbital (Dainabot, Osaka, Japan), and then injected with 0.15-0.24 μmol Gd/5-8 μL of the PAMAM-G6 contrast agent into normal mammary glands or mammary tissue surrounding a tumor (peritumorally). Dynamic micro-MR images were obtained using a 1.5-Tesla superconductive magnet unit (Signa LX, General Electric Medical System, Milwaukee, Wis.) with a birdcage type coil of 3 cm diameter fixed by a custom made coil holder. Single and double “breast coils” used for imaging breasts in humans are commercially available, for example, from GE Medical Systems (Milwaukee, Wis.) and are routinely available in outpatient MRI centers. The mice were wrapped with gauze to stabilize their body temperature and were placed at the center of the coils.

FDA-approved MR contrast agents like GPDM rarely cause serious toxicity after intravenous or subcutaneous injection. However, since adverse reactions are typically related to dose, the PAMAM-G6 contrast agent was employed at a dose that was 1/2500 of that of GPDM on a molar basis to minimize potential toxicity. Furthermore, the PAMAM-G6 agent was administered directly into the mammary gland tissue because local injection is generally safer than intravascular injection.

In order to evaluate the lymphatic drainage from the normal mammary gland of either Balb/c mice (n=5) or athymic nu/nu mice (n=5), 3D-fast spoiled gradient echo (3D-fastSPGR (efgre3d package; Signa Horizon, GE); repetition time/echo time 19.2/7.2 msec; inversion time 47 msec; 31.2 kHz, flip angle 30°, 4 excitations; 36 slice encoding steps; scan time 4 min 49 seconds) with chemical shift fat-suppression was used 6, 12, 18, 24, 30, 36, 42, and 48 min after injection of the contrast agent. The coronal images were reconstructed with 0.6-mm section thickness every 0.3-mm. The field of view was 8×4 cm and the size of the matrix was 512×256. The slice data were processed into 3D images using the maximum intensity projection (MIP) method with the same window and level (window 3500 and level 2100) (Advantage Windows, General Electric Medical System). After imaging, the mice were sacrificed by carbon dioxide inhalation.

Athymic nu/nu mice (n=4) were anesthetized and injected with 0.15 μmol Gd of GPDM into the mammary gland, and images were taken 12, 24, and 36 min after injection. Following these three images, these mice were subsequently injected with 0.15 μmol Gd of PAMAM-G6 contrast agent in the mammary gland, and images were taken at 12 and 24 min post-injection of the PAMAM-G6 contrast agent (at 48 and 60 min post-injection of GPDM).

In order to evaluate lymphatic drainage from six tumor-bearing mammary glands of three BALB-neuT transgenic mice or the PT-18 tumor-bearing mammary gland of athymic nu/nu mice (n=6), 2D fast-inversion recovery [2D-fastIR; repetition time/echo time 8000/96 msec; inversion time 150 msec; 31.2 kHz, 2 excitations; 16 slices; scan time 2 min 16 seconds] was employed to evaluate the tumor localization before injection of PAMAM-G6 contrast agent. The coronal images were reconstructed with 1.5 mm section thickness without a gap. The FOV was 8×4 cm and the size of matrix was 512×256. The slice data were processed into 3D images using the MIP method with the same window and level (window 3500 and level 2100) (Advantage Windows, GE). Then images were obtained with the 3D-fastSPGR sequence as described above.

### IV. Results

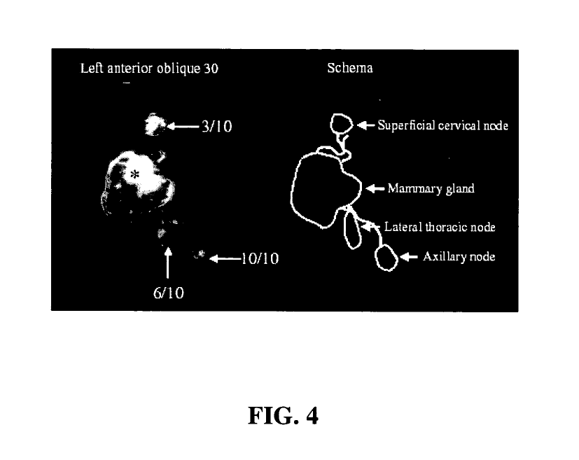

Three lymph nodes (axillary, lateral thoracic, and superficial cervical) with their draining lymphatic vessels were visualized by MRI with the PAMAM-G6 contrast agent (FIG. 4). The axillary lymph node and its afferent lymphatic vessels were visualized at the initial (6 min) time point in all 10 mice (Table 1). However, two other lymph node groups and their lymphatic vessels showed up later (Table 1). Thus, this method permitted imaging of the lymphatic system (nodes and vessels) draining normal breast tissue, and also to detect lymphatic flow over time into the cervical and lateral thoracic nodes. Table 1. Visualization of Three Major Draining Lymph Nodes (LNs) from the Normal

Breast Tissue (Visualized LNs/Total Examined LNs)

| | Time (min) | | | | | | | |
|---|---|---|---|---|---|---|---|---|
| | 6 | 12 | 18 | 24 | 30 | 36 | 42 | 48 |
| Superficial | 1/10 | 1/10 | 1/10 | 2/10 | 2/10 | 3/10 | 3/10 | 3/10 |
| cervical LN | | | | | | | | |
| Lateral thoracic | 0/10 | 1/10 | 2/10 | 4/10 | 4/10 | 5/10 | 6/10 | 6/10 |
| LN | | | | | | | | |
| Axillary | 10/10 | 10/10 | 10/10 | 10/10 | 10/10 | 10/10 | 10/10 | 10/10 |
| LN | | | | | | | | |

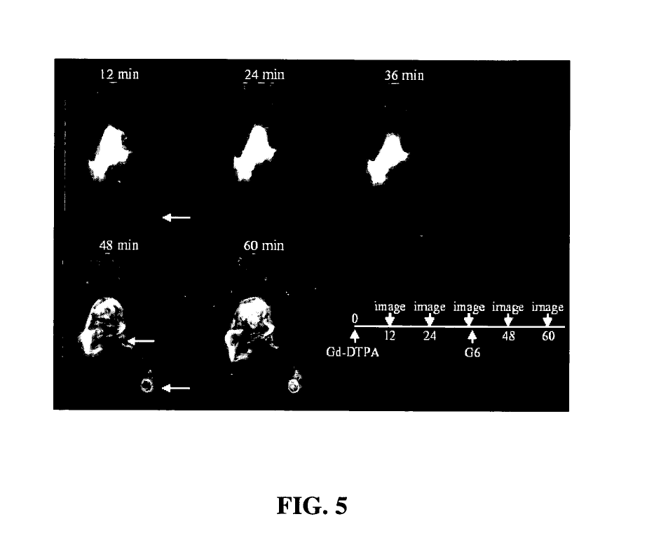

MRI images of the mice after injection of GPDM and after injection of the PAMAM-G6 contrast agent are compared in FIG. 5. Draining lymph nodes and lymphatic vessels were not well visualized at 12 minutes, 24 minutes and 36 minutes following GPDM administration. The nodes and vessels, however, were clearly visualized after administration of the PAMAM-G6 agent 36 minutes following administration of GPDM. This result demonstrates the surprisingly superior quality of images obtained with the PAMAM-G6 contrast agent compared to GPDM for visualizing the lymphatic system with MRI.

The MRI method using the PAMAM-G6 contrast agent was applied to two mouse models for breast tumors. A “spontaneous” breast cancer model using BALB-neuT transgenic mice and a PT-18 mast cell tumor xenograft injected into the breast were employed, and images of each model were obtained to visualize lymphatic drainage structure and dynamics associated with breast tumors. In both models, the flow within the draining lymphatic vessels was readily visualized (FIGS. 4 and 5).

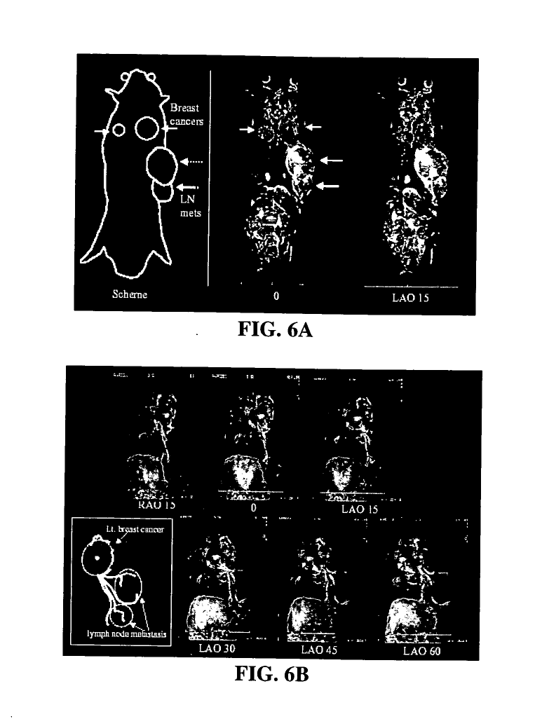

Enlarged lymph nodes containing tumor metastases and several dilated lymphatic vessels extending from the breast tumor to two tumors in lymph nodes at the lateral chest wall were clearly imaged in the BAL-neuT model (FIG. 6). FIG. 6A shows bilateral solid tumors located in the mammary glands of a mouse and associated large, metastatic lymph nodes. FIG. 6B is a series of 3D images that show several dilated lymphatic vessels extending from a breast tumor to lymph nodes in the lateral chest wall. These images demonstrate the precise localization of lymphatic structures afforded by the disclosed methods.

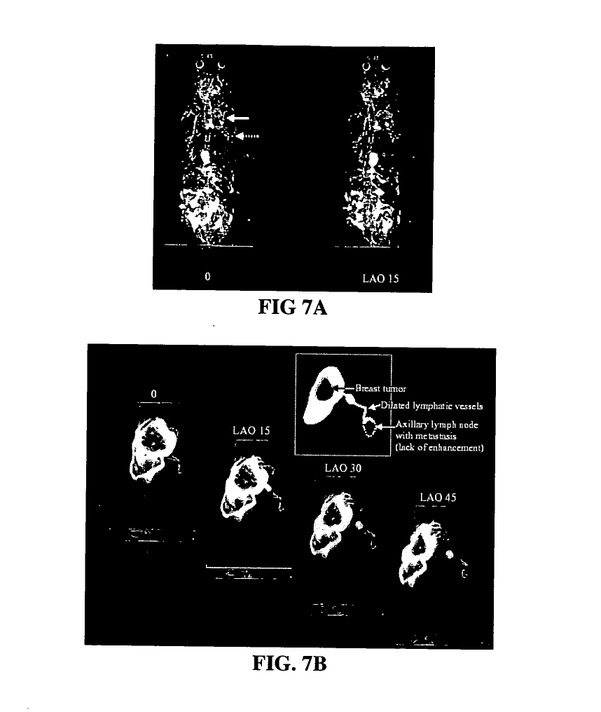

In the PT-18 model, the axillary lymph node tissue with metastatic tumor cells did not shown enhancement by the PAMAM-G6 contrast agent, but the lymphatic vessel flowing into the lymph node with a metastatic tumor was dilated and showed enhancement (FIG. 7). FIG. 7A shows a set of 2D stereo-view images of a mouse with a PT-18 tumor (solid arrow) in the breast and a tumor in the associated axillary lymph node (broken arrow). FIG. 7B shows a series of 3D images focusing (smaller field-of-view) on the breast tumor and the axillary lymph node in the PT-18 model. These images demonstrate enhancement (increase in signal intensity) of several dilated lymphatic vessels and a lack of enhancement of the interior (no increase in signal intensity) of the metastatic lymph node.

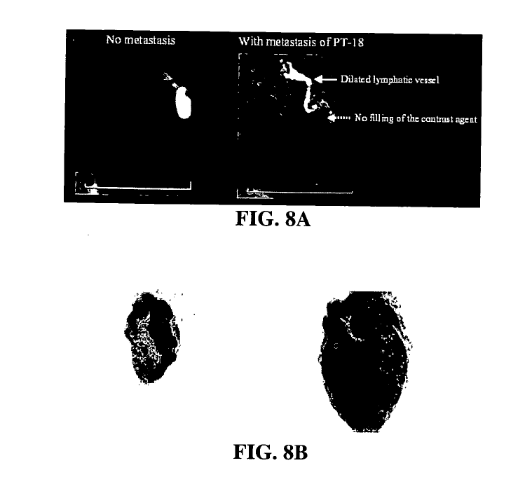

FIG. 8A shows in greater detail the differences in images (obtained using the PAMAM-G6 agent) of normal lymphatics and metastatic lymphatics in the PT-18 model. The normal lymph node is much brighter and more uniformly enhanced by the dendrimer conjugate, whereas the metastatic lymph node shows a characteristic lack of enhancement in its interior. The normal afferent lymphatic vessel of the imaged lymph node is much thinner by comparison to the dilated afferent lymphatic vessel associated with the metastatic lymph node. Dilation of the lymphatic vessel in the metastatic model is believed to be due to a blockage of lymph fluid flow through the metastatic lymph node. Histopathological examination results for the normal and metastatic lymph nodes are compared in FIG. 8B, which confirm tumor growth in the non-enhanced portions of the lymph node from the PT-18 model. All 6 mice with PT-18 tumors that were studied showed abnormalities only in the axillary nodes. The axillary node was also the predominant draining node in the tumor-bearing BALB-neuT transgenic mice. Taken together, these results are consistent with the conclusion that lymphatic flow from the mouse breast drains primarily to the axillary lymph nodes.

Sentinel lymph node localization has become a routine part of cancer surgery. Lymphoscintigraphy and intraoperative gamma probes are playing increasing roles in the surgical treatment of patients with breast cancer or malignant melanoma. However, as demonstrated in this example, MRI has potential advantages over lymphoscintigraphy. The spatial resolution of MRI (0.1-0.3 mm) is 30-100 times greater than that of scintigraphy (1 cm) and, because breast MRI utilizes surface coils that substantially decrease the field of view, the temporal resolution of MRI is greater than 10 times that of scintigraphy, offering a great potential for dynamic studies of the lymphatics. Furthermore, three-dimensional images improve anatomical localization. The absence of radiation exposure is beneficial to both surgeons and patients. Therefore, dynamic mammo-MR lymphangiography can circumvent limitations of standard lymphoscintigraphy and can help distinguish the sentinel lymph node from secondary lymph nodes.

The PAMAM-G6 contrast agent is retained by, or has an affinity for, the normal lymph node tissue, resulting in an enhanced signal in normal lymph nodes. The lack of enhancement in lymph nodes is a reliable sign for the presence of metastases. While the method might miss a lymph node entirely filled with tumor, most tumor bearing lymph nodes will have small rim (fringe) of normal tissue, which will be visualized as shown in FIG. 8.

FDA-approved MR contrast agents like GPDM rarely cause serious toxicity after intravenous or subcutaneous injection. Since adverse events are related to dose, the PAMAM-G6 contrast agent was employed in this example at a dose that was 1/2500 of that of GPDM on a molar basis to further minimize potential toxicity. Furthermore, the G6 agent was administered directly into the mammary gland tissue because local injection is generally safer than intravascular injection. In order to enhance its use for potential intraoperative localization, the PAMAM-G6 agent (and any of the other disclosed dendrimer conjugates) may be dual-labeled with gadolinium and an optical or fluorescent agent to help the surgeon to quickly and reliably localize the sentinel lymph node during surgery. Optical and fluorescent agents having reactive groups that permit easy conjugation of a colored or fluorescent dye to reactive groups on a dendrimer, such as surface amino, alcohol, and carboxyl groups, are commercially available from Molecular Probes, Eugene, Oreg. Examples of amine reactive groups include isothiocyanates, succinimidyl esters, carboxylic acids and sulfonyl chlorides. Exemplary methods for conjugating dyes to the reactive groups on the disclosed dendrimers are provided in Haughland, Molecular Probes, Inc. Handbook of Fluorescent Probes and Research Chemicals, 9^{th} ed., 2002.

In a particular embodiment, a near-IR fluorescent dye such as Cy5.5 is conjugated to the disclosed dendrimers for the purpose of optical imaging, for example, intraoperative optical imaging to help a surgeon delineate the margins of lymphatic structures. In a more particular embodiment, a G6 dendrimer that is only partially saturated with 1B4M chelating groups, leaving a number of amine groups dispersed across the surface is prepared. A Cy5.5 dye N-hydroxysuccinimidyl active ester (Amersham Biosciences, San Francisco, Calif.) is then reacted with the remaining amine groups to provide a dendrimer conjugate that can be used for optical imaging. Optical imaging using near-IR fluorescent dyes is described, for example, in Kircher et al., Cancer Res.; 63: 8122-5, 2003. Several optical imaging modalities, including fluorescence reflectance imaging (FRI) and 3D quantitative fluorescence-mediated tomography, are described in Bremer et al., Eur. Radiol.; 13: 231-43, 2002, and an optical imaging system is described in Mahmood et al., Radiology; 213: 866-70, 1999.

The disclosed MR methods using the disclosed dendrimer-based MRI contrast agents are useful in clinical practice. The particular method using the PAMAM-G6 contrast agent that was described in this Example was able to visualize both draining lymph nodes and lymphatic vessels from breast tissue in mice. This four-dimensional imaging method helped visualize the lymphatic flow over time on a 3-D display. The superior temporal and spatial resolution of this method permit wide application of the disclosed methods to the study of tumor lymphatics and lymphatic metastasis in both experimental animals and clinical medicine.

### EXAMPLE 2

### Detection of Lymphangitis and Other Lymphatic Disorders

This example compares a variety of contrast agents for MRI imaging of the deep lymphatic system and various particular components of the lymphatic system in models for a variety of lymphatic disease states.

### I. Preparation of the Contrast Agent

PAMAM-G8D, DAB-G5D, and PAMAM-G4D dendrimers (Sigma-Aldrich, St. Louis, Mo.) were each concentrated to about 5 mg/ml and diafiltrated against 0.1 M phosphate buffer at pH 9. The dendrimers were individually reacted with a 1024-, 64-, and 64-fold molar excess of 2-(p-isothiocyanatobenzyl)-6-methyl-diethylenetriamine-pentaacetic acid (1B4M), respectively, at 40° C., and maintained at pH 9 with 1 M NaOH for 24 hours. An additional equal amount of the 1B4M was added to each sample after 24 hours as a solid. The resulting preparations were purified by diafiltration using a Centricon 30 (Amicon Co., Beverly, Mass.). This resulted in over 98% of the amine groups on the dendrimers reacting with the 1B4M as determined by ¹⁵³Gd (NEN DuPont, Boston, Mass.) labeling of aliquots, as described in Example 1.

Subsequently, about 3 mg of each dendrimer-1B4M conjugate (containing 4 μmol 1B4M) was mixed with 8 μmol of non-radioactive Gd(II) citrate in 0.3 M citrate buffer, pH 4.5, overnight at 40° C. The excess Gd(III) in each preparation was removed by diafiltration using a Centricon 30 filter (Amicon Co.) while simultaneously changing the buffer to 0.05 M PBS. The purified samples were diluted to 0.2 mL with 0.05 M PBS and about 5 μL was used in each mouse extremity. A replacement assay using ¹⁵³Gd was used to determine that 80%, 85%, and 84% of the 1B4M on the PAMAM-G8, DAB-G5, and PAMAM-G4 dendrimer-1B4M conjugates, respectively, was indeed chelating Gd(III) ions (see Example 1). In brief, approximately 300,000 cpm of ¹⁵³Gd were added with 0.1 mmol of nonradioactive Gd(III) to 5 mL of the injected samples. The samples were then incubated in 0.5 M citrate buffer for 2 hours at 40° C., after which the bound and unbound fractions were separated using a PD-10 column (Pfizer, Providence R.I.).

Commercially available Gadomer-17 and GPDM (Schering AG, Berlin, Germany) were obtained and compared to the disclosed dendrimer conjugates.

### II. Preparation of the Mice

To generate a lymphangitis model, Concanavalin A (300 mg) (Sigma, St. Louis, Mo.) was injected intravenously into C57BL6 mice (NCI, Frederick, Md.) 24 hours before MRI was performed. Histological analysis of this mouse model demonstrated massive lymphocytic infiltration of most of the organs and tissues, especially in the liver, where cavernous dilation of lymphatic vessels was observed.

Eight- to 10-month old IL-15 transgenic mice (on a C57BL6 background) were used as a chronic lymphoproliferative/neoplastic disease model because they manifested selective expansion of NK, CD8⁺NK-T, γδIELs, and CD8 T cells in the periphery. Most of the lymph nodes collected from an aged IL-15 transgenic mouse were enlarged in size. The lesion of proliferated monoclonal lymphocytes often involved the lung and the subcutaneous lymphatic tissue. Pathological analysis of a lymph node from the IL-15 transgenic mouse revealed massive lymphocytic infiltration, as demonstrated by hematoylin and eosin staining. Interestingly, a typical germinal center structure was no longer seen within the lymph nodes.

In addition, 10-week-old C57BL6 mice (NCI, Frederick, Md.) bearing lymph node metastases of MC38 colorectal cancer cells following either intravenous or subcutaneous injection were used as a mouse model manifesting lymph node metastasis.

### III. Injection and Imaging

The mice were anesthetized with 1.15 mg sodium pentobarbital (Dainabot, Osaka, Japan) by intraperitoneal injection, and then injected intracutaneously with 0.1 μmolGd of PAMAM-G8, DAB-G5, PAMAM-G4, Gadomer-17, or GPDM into four middle phalanges in all four extremities. Dynamic micro-MR images were obtained using a 1.5-Tesla superconductive magnet unit (Signa LX, General Electric Medical System, Milwaukee, Wis.) with a round birdcage type coil of 3 cm diameter fixed by a custom-made coil holder. Four to eight female mice (7 weeks old, 18-21 g body weight) in each group were used and each contrast agent was prepared at least three separate times for these imaging studies. The mice were wrapped with gauze to stabilize their body temperature and were placed at the center of the coils.

A 3D-fast spoiled gradient echo (3D-fastSPGR (efgre3d package; Signa Horizon, GE); repetition time/echo time 28.5/7.9 msec; inversion time 65 msec; 31.2 kHz, flip angle 30°, four excitations; 40 slice-encoding steps; scan time 7 min 36 seconds) with chemical shift fat-suppression was used 10, 20, 30, and 45 min after injection of the contrast agents. The coronal images were reconstructed with 0.6-mm section thickness at every 0.3-mm. The field of view was 6×3 cm and the size of the matrix was 512×256. The intensities of the regions of interest (for example, a whole axillary lymph node, and the liver) were measured, and then the time-intensity curves analyzed. The data were expressed as the axillary lymph node-to-muscle and the axillary lymph node-to-liver ratios. The slice data were processed into 3D images using the MIP method with the same window and level (window 3500 and level 2300) (Advantage Windows, General Electric Medical System). Two board-certified radiologists separately read a set of stereo views of 3D-MRL for all studies, and estimated the visualization of the thoracic duct. After imaging, the mice were sacrificed by carbon dioxide inhalation.

Enlarged lymph nodes that clearly contained abnormal lesions, as demonstrated by MRL, were isolated from the mouse following MRL image acquisition using flow cytometry. After the lymphocytes were purified by Ficoll density separation, the cells were first incubated with an anti-CD16 antibody (Pharmingen, San Diego, Calif.) to block FcγR-mediated staining, and then stained with a combination of FITC-anti CD3 (Pharmingen), Phycoerythrin-NK1.1 (Pharmingen), and Cychrome-anti CD8 (Pharmingen). They were incubated for 15 minutes at room temperature.

Statistical analyses were performed using either Student's t-test or a one-way analysis of variance (ANOVA), with a pairwise comparison using the Bonferroni method for signal intensity curves (Statview; SAS Institute Inc., Cary, N.C.).

### IV. Results

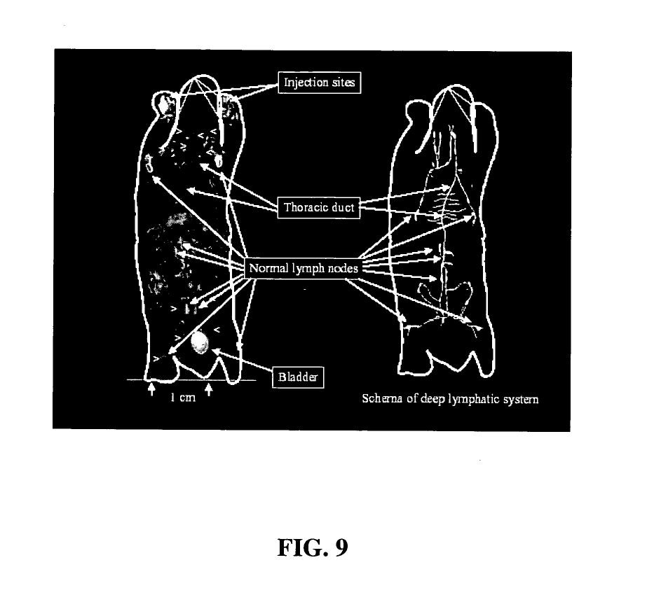

As shown in FIG. 9, the PAMAM-G8 contrast agent enabled most of the deep lymph nodes to be visualized in a mouse. The schema shown in FIG. 9 shows that a number of normal lymph nodes and the thoracic duct were imaged following injection of the PAMAM-G8 contrast agent intradermally into the extremities of the mouse.

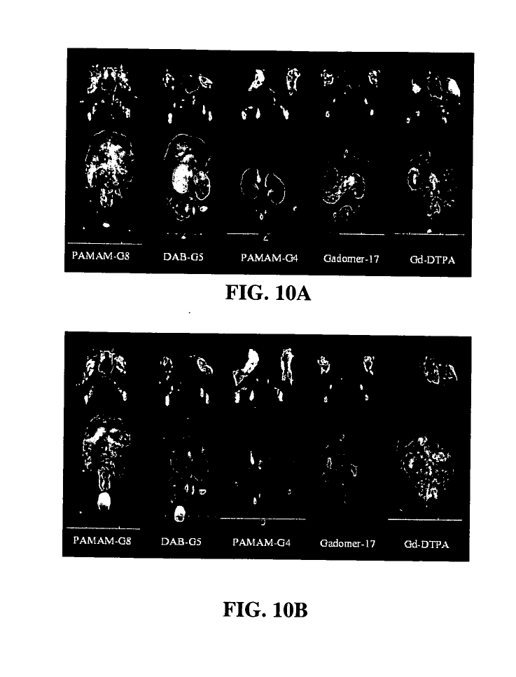

A comparison of the images obtained using the PAMAM-G8, PAMAM-G4, and DAB-G5 contrast agents to images obtained using Gadomer-17 and GPDM is shown in FIG. 10. FIG. 10A compares the images obtained using these agents 10 minutes after injection. The lymph nodes (particularly those about ⅔ of the way up the body of the mice) appear much brighter and well-defined in the images obtained using the dendrimer conjugates than in the images obtained with Gadomer-17 and GPDM. As shown in FIG. 10B, the dendrimer conjugates exhibit persistent superior image contrast and detail of the lymphatic system 45 minutes after injection in comparison to Gadomer-17 and GPDM. Virtually no image enhancement is observed after 45 minutes using GPDM. Most of the deep lymph nodes were visualized throughout the mouse at all time points examined using the dendrimer conjugates. Gadomer-17 allowed visualization of the deep lymph nodes, albeit not as clearly and brightly as the dendrimer-based agents. In contrast, GPDM did not allow most of the lymph nodes to be visualized even at early times.

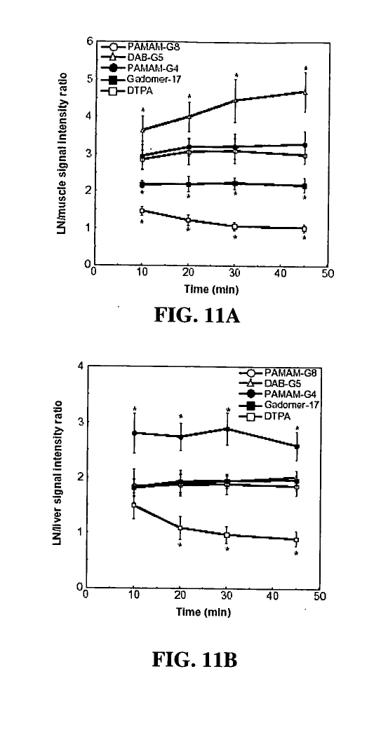

To compare the clarity of the images with different contrast agents in a semi-quantitative fashion, the ratio between the intensity of signals (T₁-weighted signal) obtained from the axillary lymph node and that from the neighboring muscle tissue (the “background”) was calculated at different times following administration. The results are shown in FIG. 11A. The axillary lymph node-to-background ratio obtained with DAB-G5 was significantly higher than that obtained with either PAMAM-G8 or PAMAM-G4 at all time points examined (P<0.01). The axillary lymph node-to-background ratio obtained with PAMAM-G8 or PAMAM-G4 was significantly higher than that measured with Gadomer-17 and GPDM at all time points examined. Furthermore, the axillary lymph node-to-background ratio obtained with Gadomer-17 was significantly higher than that acquired with GPDM (P<0.01). These results illustrate the superior image quality obtained with a number of the disclosed dendrimer conjugates.

To further compare the ability of the five contrast agents to aid visualization (clarity of contrast) of lymph nodes close to the organs responsible for the excretion of the contrast agents, signal intensity ratios were then measured between the signal at the axillary lymph node and the signal at the liver (FIG. 11B). The axillary lymph node-to-liver ratio obtained with PAMAM-G4 was significantly higher than that acquired with PAMAM-G8, DAB G-5, or Gadomer-17 at all time points examined (P<0.01). The axillary lymph node-to-liver ratios of PAMAM-G8, DAB-G5, or Gadomer-17 were nearly equivalent, but were significantly higher than that measured with GPDM (P<0.01).

The lymphatic vessels were better visualized with PAMAM-G8 compared to all other agents examined, followed by the PAMAM-G4 agent. In particular the PAMAM-G8 contrast agent permitted visualization by a radiologist of the thoracic duct in all mice at all times after administration. The ability of the contrast agents used in this Example to provide sufficient contrast to allow identification by radiologists of the thoracic duct are summarized in Table 2 below.

TABLE 2
Visualization of Thoracic Duct on the Dynamic Study
of MRL (Number of Mice in Which the Thoracic Duct was
Visualized/Number of Mice Examined)

Time (min)

| | 10 | 20 | 30 | 45 |
|---|---|---|---|---|
| PAMAM-G8/Normal Mice | 10/10 | 10/10 | 10/10 | 10/10 |
| DAB-G5/Normal Mice | 3/5 | 2/5 | 0/5 | 0/5 |
| PAMAM-G4/Normal Mice | 4/5 | 4/5 | 3/5 | 2/5 |
| Gadomer-17/Normal Mice | 2/5 | 2/5 | 1/5 | 1/5 |
| GPDM/Normal Mice | 2/5 | 0/5 | 0/5 | 0/5 |

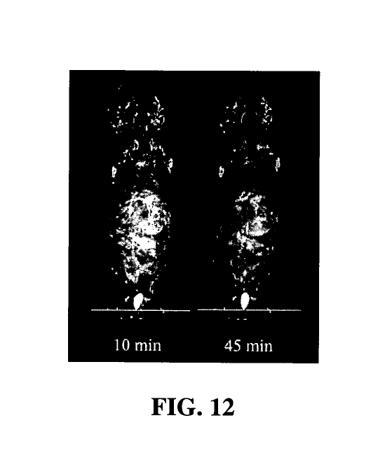

The five contrast agents were next evaluated for their ability to visualize the status of diseases associated with the lymphatic system in three different mouse models. Lymphangitis was induced in mice by injecting Conocanavin A intravenously, as typically accompanies systemic dilatation of lymphatic vessels. As shown in FIG. 12, dynamic 3D-micro-MRL with PAMAM-G8 demonstrated the remarkable dilation of the lymphatic vessels throughout the body, especially in the liver. Enhanced structures were mostly shown by 2D-micro-MRL along vessels, including hepatic veins (data not shown). Those structures correlated well with the dilated lymphatic vessels on histological specimens.

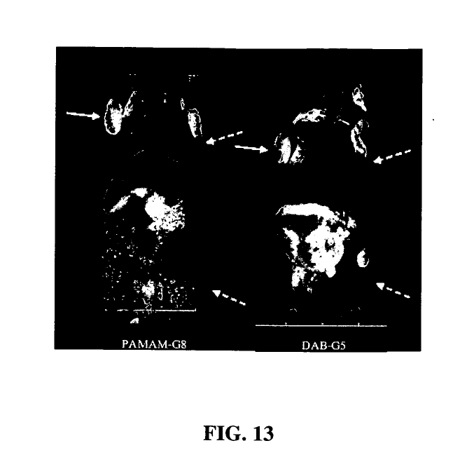

Mice with lymphoproliferation/lymphoma were also examined. The IL-15 transgenic mice that were used develop manifested polyclonal expansion of NK, NK-T, γδ-T, and memory phenotype CD8⁺ T-cells in the periphery, and macroscopic examination demonstrated enlarged spleen and lymph nodes (data not shown). Dynamic 3D-micro-MRL, FIG. 13, demonstrated considerable multiple lymph node swellings with central filling defects (non-enhancing area), when either PAMAM-G8 or DAB-G5 was used as the contrast agent (left and right images, respectively). In these images, taken 45 minutes after injection of the respective contrast agents, dilation of subcutaneous lymphatic vessels (broken arrows) and swollen right axillary lymph nodes (solid arrows) are indicated. As seen, however, dilation of lymphatic vessels in these mice was best visualized with PAMAM-G8 (left image). Pathological examination of the swollen lymph nodes showing filling defects yielded results consistent with the micro-MRL observations. In brief, an accumulation of homogenous, dense lymphoid cells in major lymph nodes from aged (>32 wk) IL-15 transgenic mice were observed, indicating that these mice manifested a chronic lymphoproliferative status. In particular, the germinal center structure was no longer seen in these lymph nodes. Furthermore, immunological examination revealed that these cells were mostly mature CD8⁺ T-cells. The densely packed infiltrating CD8⁺ T-cells within the lymph node appeared to form a tight boundary, preventing free penetration of the macromolecular contrast agent. Although polyclonal expansion of NK, NK-T, and γδ-T IELs lymphocytes were observed in the IL-15 transgenic mouse, these cells did not manifest chronic expansion or infiltration into the lymph nodes. These observations collectively demonstrate that the IL-15 transgenic mice develop chronic CD8⁺ T cell expansion/proliferation in multiple lymph nodes with aging, which may lead to the onset of a lethal pathological condition (such as lymphoma).

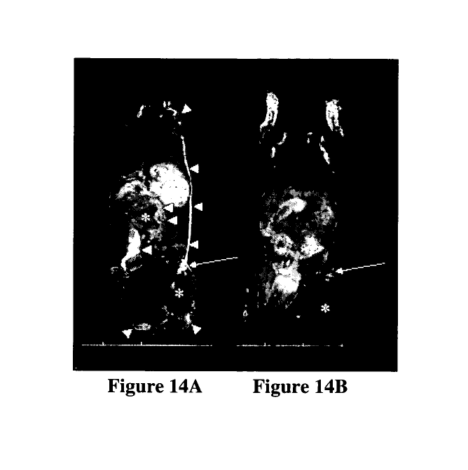

The potential of 3D-MRL with PAMAM-G8 to examine lymph nodes infiltrated with non-lymphoid cells also was examined. MC-38 colorectal cancer cells form metastatic growths in lymph nodes of syngenic C57BL6 mice following intravenous injection, and thus provide an appropriate model in which to evaluate this system. A 3D image of a mouse with a lymph node metastasis obtained 45 minutes after injection of the PAMAM-G8 agent is shown in FIG. 14 (left image). Large inguinal and abdominal tumors (asterisks) accompanied by the left inguinal lymph node (long arrow) are seen, along with dilated lymphatic vessels surrounding the tumor and a collateral lymphatic vessel, which communicated with the thoracic duct via the axillary lymph node (arrowheads). In contrast, mice with a subcutaneously xenographed MC-38 tumor in the same location did not show any abnormalities in either the lymph nodes or the lymphatic vessels by 3D-MRL with PAMAM-G8 (right image), confirming that the growth of tumor cells in the lymph node tissue specifically caused the abnormal image characteristics visualized by this method.

The effectiveness of the disclosed methods in diagnosing and differentiating various lymphatic disease by visualizing abnormally developing lymph nodes and lymphatic vessels associated with inflammation, proliferative disorder, and tumor metastasis were demonstrated in this example. In addition, each of the dendrimer-based contrast agents exhibited distinct characteristics, which may be exploited for different purposes in clinical applications. For example, PAMAM-G8 appears to be better suited for imaging of lymphatic vessels and diverse other components of the lymphatic system, whereas DAB-G5 may be better suited for imaging of lymph nodes. PAMAM-G4 appears to be particularly suited for visualization of abdominal lymph nodes adjacent to the liver based on its high lymph node to liver signal intensity.

In summary, the disclosed dendrimer conjugates are superior contrast agents for imaging the lymphatic system in comparison to Gadomer-17 and GPDM, even at lower dosages warranted by the potential toxicity of metal ions, such as gadolinium ions, that may be released from the disclosed dendrimer conjugates.

### EXAMPLE 3

### Comparison of PAMAM-G8 and GPDM for Detecting and Differentiating Lymphatic Disorders Including Infection and Metastatic Conditions

This example further demonstrates the ability of the disclosed methods to assess and differentiate differing disease states of the lymphatic system. In this example, the PAMAM-G8 contrast agent is compared to GPDM. PAMAM-G8 was prepared as described in Example 2, and GPDM was purchased (Schering AG, Berlin, Germany). Animal models, administration of the contrast agents, and 3D-micro-MRL were as described in Example 2.

### I. Results

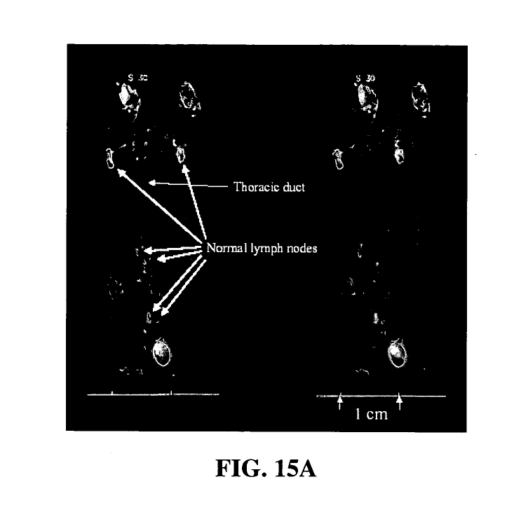

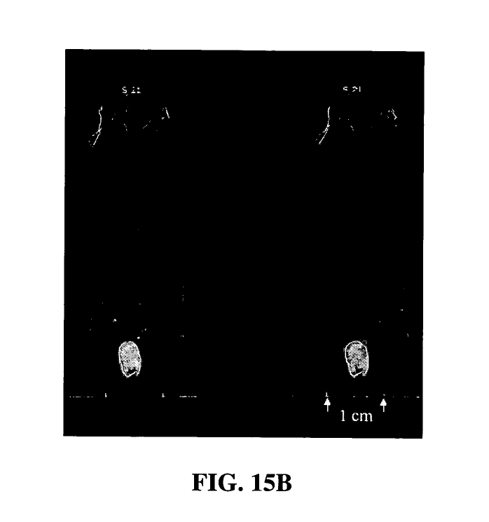

As seen in FIG. 15, most deep lymph nodes throughout the body were visualized by 3D-microMR-lymphangiography with PAMAM-G8 (FIG. 15A), but not with GPDM (FIG. 15B). In addition, the thoracic duct was visualized in all six mice that were given injections of PAMAM-G8, but not in mice given injections of GPDM. Other lymphatic vessels were also better visualized with PAMAM-G8 than with GPDM.

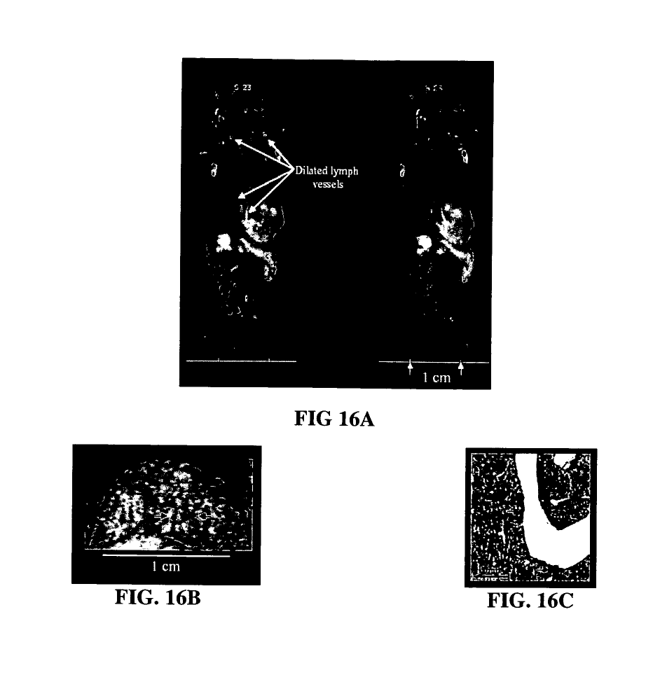

Concanavilin A was injected into normal mice to induce lymphangitis. As shown in FIG. 16A, three-dimensional micro-MRL using PAMAM-G8 was able to detect remarkable dilation of lymphatic vessels (arrows) throughout the whole body, and especially in the liver. The enhancement in the liver was seen adjacent to the vascular structure, but the vascular structures themselves did not show enhancement (arrows in FIG. 16B). Histology analysis (FIG. 16C) revealed that the lymphocytes had mainly infiltrated adjacent to the vascular structures with cavernous dilation (>10 μm) of lymphatic ducts filled with massive lymphocytes (arrows), corresponding to the enhancement location and consistent with the imaging results.

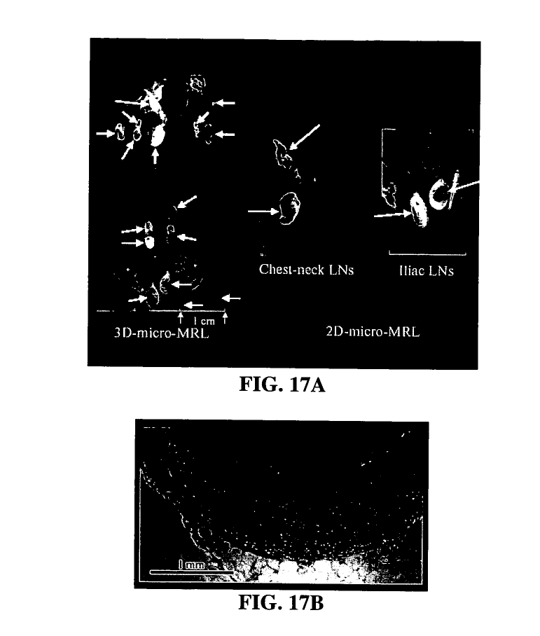

Lymph node changes in a proliferative or neoplastic model also were evaluated. FIG. 17A shows a series of images taken of a IL-15 transgenic mice which showed considerable lymph node swelling (3D image) with non-enhancing central filling defects (2D-images). The abnormal lymph nodes identified by micro-MRL were targeted for removal, living cell sampling and subsequent analysis. Immunological and molecular biological analyses to demonstrate the cellular phenotypes, the receptor expressions, and the clonality of the infiltrative cells in individual mice also were performed. These pathological examinations confirmed that the observation of filling defects in the images were due to replacement of the germinal center structure of lymph nodes by a homogeneous dense infiltration of lymphoid cells that restricted access of the contrast agent (as shown in the histological image of FIG. 17B). These observations collectively demonstrate that with age the L-15 transgenic mouse develops CD8⁺ T-cell expansion and proliferation in multiple lymph nodes, which may lead to the onset of lethal pathological conditions such as lymphoma.

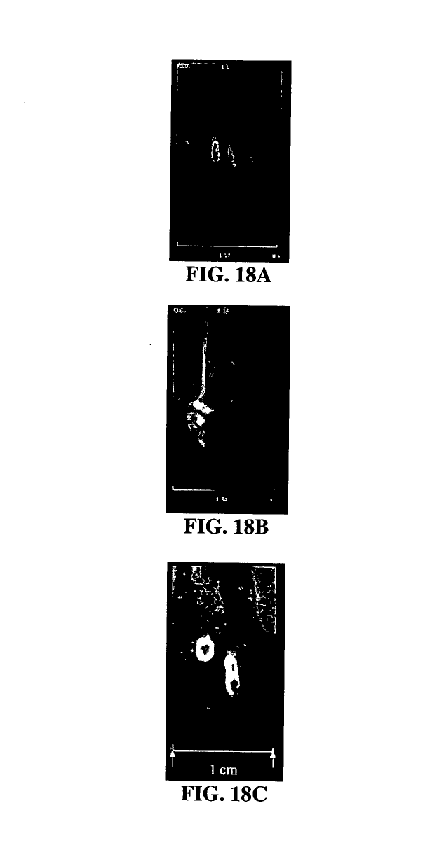

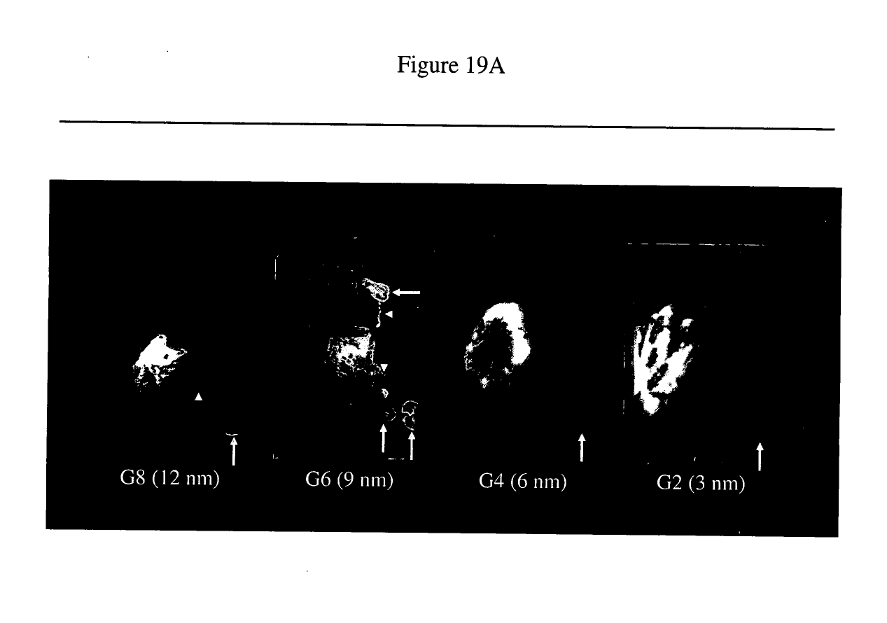

Nude mice that develop spontaneous oral ulcers and urinary tract infections also were examined. Images taken 45 minutes after injection with PAMAM-G8 in this infection model, showed an irregular dilation of the lymphatic vessels in the lymph nodes (FIG. 18B). These enlarged infections lymph nodes can be differentiated in images from normal lymph nodes (FIG. 18A, also obtained with PAMAM-G8), in which small lymph nodes are observed, and from enlarged lymph nodes in the metastatic IL-15 transgenic mouse model (FIG. 18C, also obtained with PAMAM-G8), which exhibit central filing defects characteristic of the presence of metastatic cells in the lymph node.

The MRL methods described herein are applicable to both investigative studies in laboratory animals and in clinical practice with human subjects. With respect to micro-MRL in mice, the disclosed methods permit detection of abnormalities in the lymphatic system throughout the whole body in a live animal, allowing evaluation of time-dependent changes in the same mouse (data not shown). The methods also permitted targeted removal, and subsequent analysis, of involved lymph nodes in IL-15 transgenic mice with lymphoadenopathy. Since immunological and molecular biological analyses demonstrated the cellular phenotypes, the receptor express, and the clonality of the infiltrative cells, the results will be diagnostically useful in determining the consequences of the expantion of CD8⁺ T-lymphocytes in IL-15 transgenic mice.

The dilated liver and mesenteric lymphatic systems were enhanced and visualized in the concanavalin A-induced lymphangitis model by this method. The liver lymphatic enhancement was found just surrounding the vasculature in the disease model mice. The enhancement tended to locate along the hepatic veins as shown in FIG. 16. Therefore, an amount of contrast agent which attached to lymphocytes, could migrate from the main trunk of the lymphatic vessels back to liver or mesenteric systems associated with lymphocyte infiltration. Thus, it might enhance the liver and mesenteric system lymphatic systems, especially under the condition of lymphatic congestion.

In conclusion, micro-MRL with the PAMAM-G8 contrast agent was able to visualize most of the lymph nodes throughout the body and could distinguish infections expansion of lymphocytes from that caused by chronic lymphoproliferative conditions.

### EXAMPLE 4

### Preparation of Paramagnetic Contrast Agents and Study of their Ability to Image the Lymphatic System

Agents with different chemical properties, including a low molecular weight FDA-approved extracellular MRI contrast agent, Gd-[DTPA]-dimeglumine (Magnevist®, MW=938 Da) (Schering AG, Berlin, Germany), Gadomer-17 (MW=17 kD, Schering AG, Berlin, Germany), and a DAB-G5 contrast agent, were compared with certain PAMAM dendrimer-based agents.

### I. Preparation of the Contrast Agent

The generation-2 (G2), -4 (G4), -6 (G6), and -8 (G8) polyamidoamine (PAMAM) dendrimers (Aldrich Chemical Co., Milwaukee, Wis.) have an ethylenediamine core, with 16, 64, 256, and 1024 terminal reactive amino groups, and a molecular weight of 3,256, 14,215, 58,048, and 233,383 Da, respectively. The generation-S polypropylenimine dendrimer (DAB-G5) has a diaminobutane core, 64 terminal reactive amino groups, and a molecular weight of 7,168 Da.

Dendrimers were concentrated to ˜5 mg/ml and diafiltrated against 0.1 M phosphate buffer at pH 9. PAMAM-G2, -G4, -G6, -G8, and DAB-G5 dendrimers were reacted with a 24-, 96-, 256-, 1024-, and 64-fold molar excess of 2-(p-isothiocyanatobenzyl)-6-methyl-diethylenetriamine-pentaacetic acid (1B4M) at 40° C., respectively, and maintained at pH 9 with 1 M NaOH for 24 hr. An additional equal amount of the 1B4M was added as a solid after 24 hours. The resulting preparations were purified by diafiltration using a Centricon 10 (Amicon Co., Beverly, Mass.) for G2 and a Centricon 30 (Amicon Co.) for G4, G6, and G8.

Subsequently, all dendrimer-1B4M conjugates (˜3 mg containing 4 μmol 1B4M) were mixed with 8 μmol of non-radioactive Gd(III) citrate in 0.3 M citrate buffer overnight at 40° C. The excess Gd(III) in the preparations was removed by diafiltration using a Centricon 10 for G2 and DAB-G5 and Centricon 30 filter for G4, G6, and G8 while simultaneously changing the buffer to 0.05 M PBS. Previously reported quality control methods were performed to determine the degree of chelation on each dendrimer generation. Kobayashi et al., J. Nat'l Cancer Inst.; 96(9):703, 2004.

### II. Preparation of the Mice

All mouse studies were approved by the Animal Care and Use Committee of the National Institutes of Health. Ten week-old athymic nu/nu mice (n=5 in each group) (NCI, Frederick, Md.) were used for each arm of the study.

Tumor xenografts were created by injecting 10⁷ PT-18 cells, a murine mast cell line, into the left mammary pad in athymic nu/nu mice. Tumors of 4-10 mm developed in the mammary pad within 15 days of injection. Although 2 of 7 left axillary lymph nodes were palpable, PT-18 cells infiltrated into 6 of 7 lymph nodes on histological specimens.

### III. Dynamic 3D-MR Lymphangiography

Mice were anesthetized with 1.15 mg sodium pentobarbital (Dainabot, Osaka, Japan) by intraperitoneal injection. Then 0.15 μmolGd/5 μL of all nano-size contrast agents or 0.45 μmolGd/5 μL of Gd-[DTPA]-dimeglumine were injected into normal mammary glands or mammary tissue surrounding a tumor. Dynamic MR images were obtained using a 1.5-Tesla superconductive magnet unit (Signa LX, General Electric Medical System, Milwaukee, Wis.) with a birdcage type coil of 3 cm diameter fixed by a custom-made coil holder. The mice were wrapped with gauze to conserve their body temperature and were placed at the center of the coils.

Effect of Contrast Agent Size on Imaging

In order to evaluate the effect of contrast agent molecular size on the ability to visualize the lymphatics and lymph nodes of athymic nu/nu mice, thirty five non-tumor bearing nu/nu mice were divided into seven groups (n=5). The mice were evaluated with 3D-fast spoiled gradient echo (3D-fastSPGR (efgre3d package; Signa Horizon, GE); repetition time/echo time 19.2/7.2 msec; inversion time 47 msec; bandwidth 31.2 kHz, flip angle 30°, 4 excitations; 36 slice encoding steps; scan time 4 minutes 49 seconds) with chemical shift fat-suppression at 6, 12, 18, 24, 30, and 36 minutes post-injection of each contrast agent. The coronal images were reconstructed with 0.6-mm section thickness every 0.3-mm. The field of view was 8×4 cm and the size of the matrix was 512×256. The slice data were processed into 3D images using the maximum intensity projection (MIP) method with the same window and level (window 3500 and level 2100) (Advantage Windows, General Electric Medical System). The enhancement ratio was calculated by taking the average signal intensity in the axillary lymph node and dividing it by the signal intensity in the adjacent muscle.

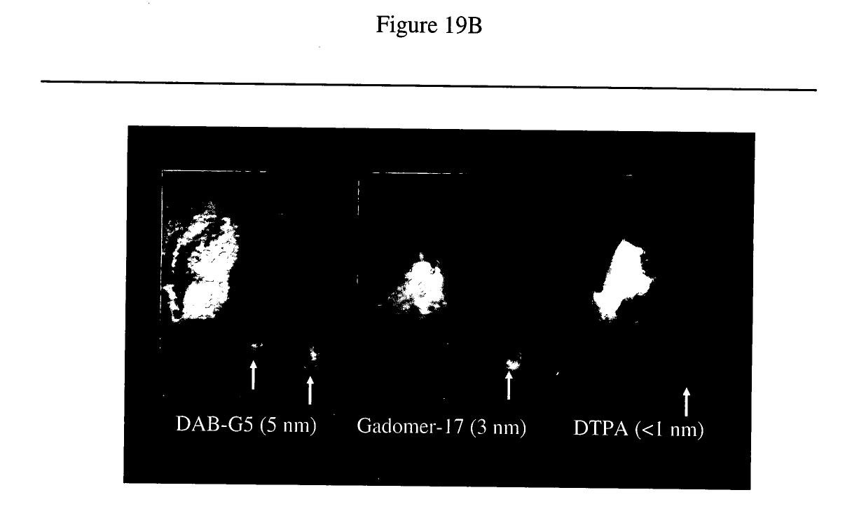

G6 nanoparticle produced the earliest and most intense opacification of the sentinel lymph nodes. MRL employing the G6 contrast agent (˜9 nm) depicted the axillary lymph nodes and their lymphatic vessels more clearly than the other gadolinium-based agents compared in this example (FIGS. 19A and 19B). Among all the PAMAM dendrimer-based agents utilized in this example, the axillary lymph nodes and their lymphatic vessels were visualized at all time points in all 10 mice with the G8 (˜12 nm) dendrimer conjugate and the G6 (˜9 nm) dendrimer conjugate. The G6 (˜9 nm) dendrimer conjugate yielded higher signal intensity within the axillary lymph node compared to the G8 agent (˜12 nm) at the two initial time points (2.6±0.2 vs 1.7±0.2 (p<0.001), 2.9±0.3 vs 2.3±0.2 (P=0.006) for G6 vs G8 at 6 and 12 minutes, respectively) but thereafter were equivalent (FIG. 19A). However, neither the G4 (˜6 nm) nor the G2 (˜3 nm) dendrimer conjugate satisfactorily opacified the lymph nodes; they were able to depict the lymph nodes only faintly and then only up to 12 minutes post-injection. Thus, the G6 dendrimer conjugate was the best agent to visualize axillary lymphatic vessels and lymph nodes in this model.

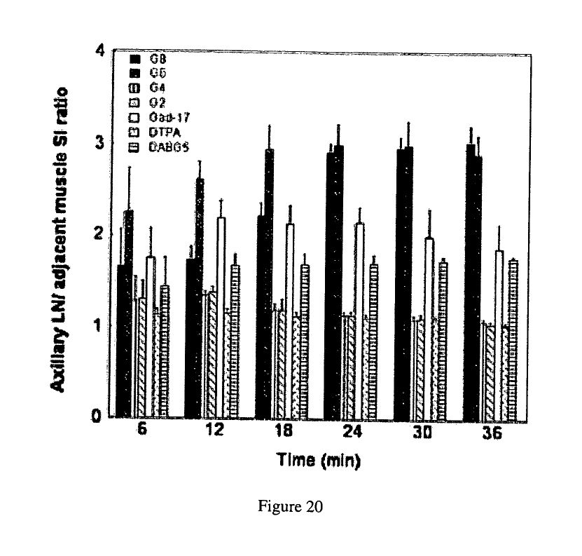

While they did not visualize the axillary lymph nodes as well as the G6 and G8 dendrimers, Gadomer-17 and DAB-G5 dendrimers were superior to the G2 and G4 PAMAM dendrimers. Gadomer-17 and DAB-G5 dendrimers have similar elution time by size-exclusion HPLC to the G2 and G4 dendrimers, respectively, but were more effective for visualizing lymph nodes. This may relate to the higher hydrophilicity of the PAMAM dendrimers (FIGS. 19B and 20). Gd-DTPA failed to opacify either the lymph nodes or the lymphatic vessels (FIGS. 19B and 20). In comparison to the G6 and G8 dendrimers, G2, G4, DAB-G5, Gadomer 17, and Gd-DTPA yielded lower signal intensities within the axillary lymph nodes while the signal intensity of adjacent muscle with the G2, G4, Gadomer-17, and Gd-DTPA was significantly higher than those measured with G6 and G8 (p<0.05).

Correlation of T1 MRI Signal Intensity to G6 Agent Concentration

In order to verify the correlation between T1-weighted MRI signal intensity and concentration of G6 agent, serum phantoms were made containing various concentrations of the G6 agent with a bovine serum (GIBCO, Invitrogen Co., Carlsbad, Calif.) and MR images were obtained with the same imaging technique as shown above. Three sets of phantoms, high (0.1-40 mMGd), intermediate (0.1-1 mMGd) and low (0.01-0.1 mMGd) concentrations, were studied. A sample of Gd-DTPA was used as an internal control.

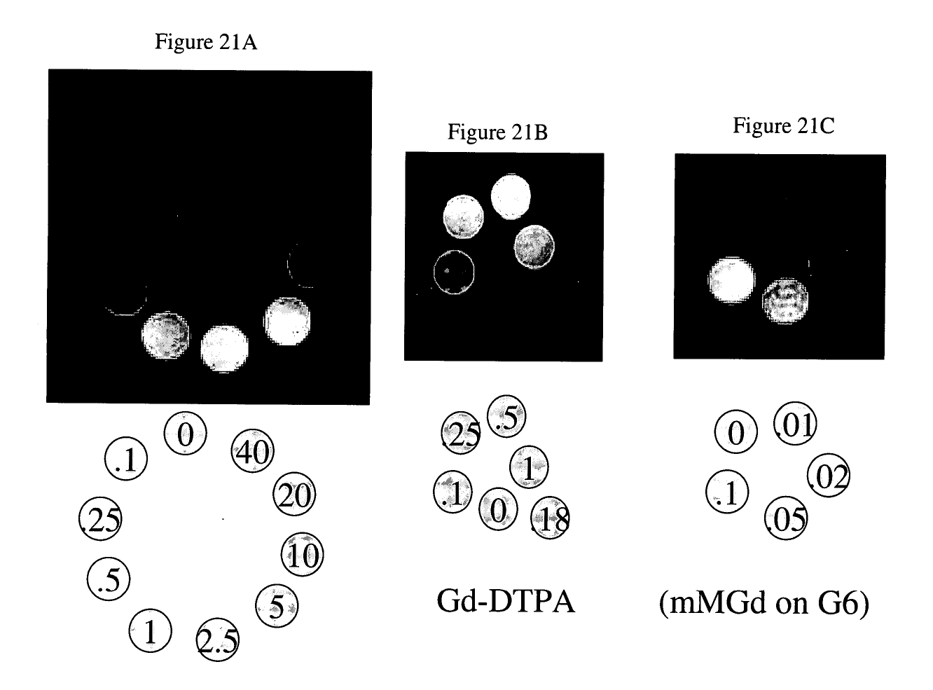

Increased T1-weighted MRI signals were detected as G6 dendrimer conjugate concentration increased, ranging from 0.02 mMGd to 5 mMGd. When compared to the concentration of G6 dendrimer conjugate in phantoms, the G6 agent was detected at concentrations ranging from 0.02 mM Gd to 5 mMGd (FIG. 21C). T1-weighted MRI signal increased as concentration of G6 agent increased up to a concentration of 5 mM Gd (FIG. 21A). Beyond this concentration no further increases in signal were seen due to T2* effect and T1-saturation at >5 mMGd. G6 dendrimer conjugate has a much higher T1 relaxivity than Gd-DTPA (FIG. 21B) because it is a macromolecule.

Quantification of Contrast Agent in Sentinel Lymph Nodes

In order to semi-quantify the concentration of contrast agent within each sentinel lymph node, non-tumor bearing athymic nu/nu mice (n=8) were anesthetized and injected with 0.40 μmolGd of the G6 contrast agent (40 mMGd/6400 ppm) into the mammary gland, and axial images were taken with serum phantoms containing known concentrations (2.5 and 5 mM) of G6 using the 3D-fastSPGR (repetition time/echo time 19.2/7.2 msec; inversion time 47 msec; bandwidth 31.2 kHz, flip angle 30°, 3 excitations; 16 slice encoding steps; scan time 2 minutes 25 seconds) 12, 24, 36, 48, and 60 minutes after injection.

To validate the concentration of G6 agent, we used another method to calculate the Gd concentration in the enhanced left axillary lymph node. The consecutive MR images of 5 separate mice together with the same phantoms used above were taken with the same 3D-fSPGR protocol using 4 flip angles (10, 20, 30, and 40 degrees) at 24 min post-injection of 40 mM/20 μL G6 agent in the left mammary gland. Signal intensity of the enhanced left axillary lymph node was corrected with that in the right axillary lymph node as a non-enhanced control. Then, signal intensities obtained with different flip angles were plotted in order to calculate decreased T1 values in the left axillary lymph node due to the presence of G6 agent. Gd(III) concentrations were calculated based on the decreased T1 values in the enhanced node and the R1 relaxivity of G6 agent at this condition. Technically, multi-flip angle scans were able to be performed once an hour because of the gradient driver overheat on the MRI system. In order to calculate serial concentration of the G6 agent, this separate set of mice was used only to obtain the T1 values. The serial concentrations of G6 agent were calculated based on serial scan data under flip angle (30°) and T1 values obtained from multi-flip angle scans.

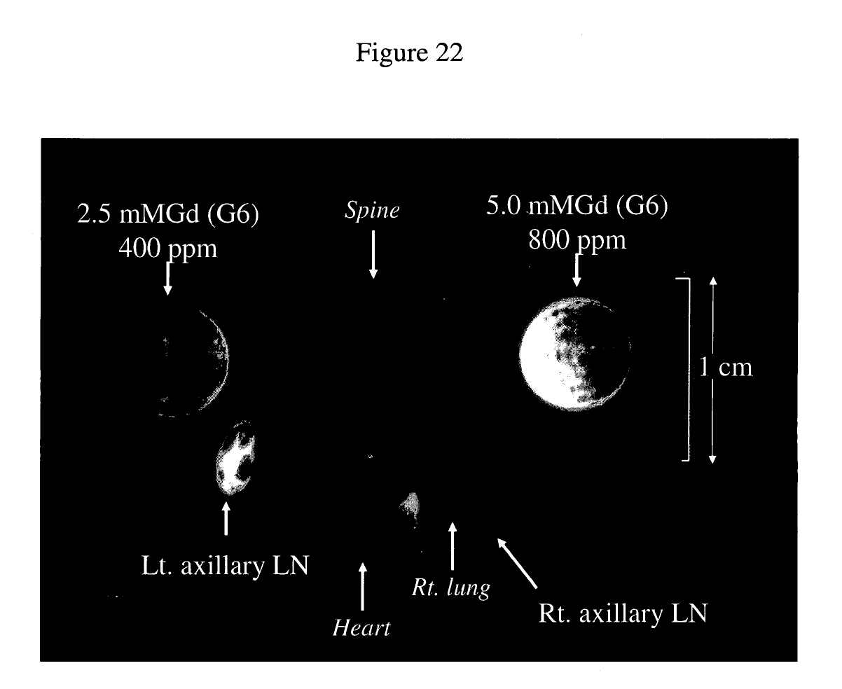

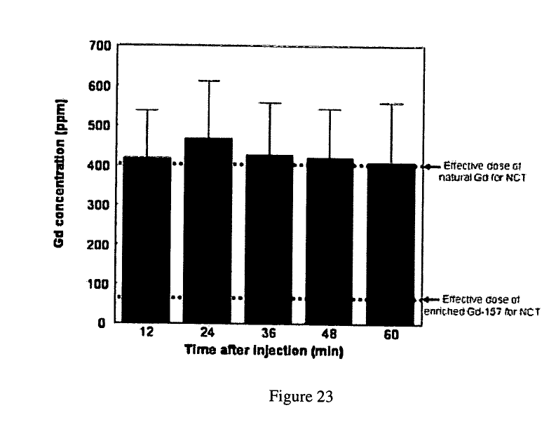

A sufficient concentration of gadolinium for gadolinium NCT can be delivered to the sentinel lymph nodes using a G6 contrast agent. Based on MRL with injection of 40 mMGd/6400 ppm of G6 dendrimer conjugate, the concentration of the G6 agent in the axillary lymph nodes ranged from 276 to 683 ppm (1.7 to 4.3 mMGd) for up to 60 minutes post-injection in five mice tested (FIGS. 22 and 23). The maximum concentrations of G6 agent were found at 24 minutes post-injection in two of five mice and 36 minutes post-injection in three of five mice. The average maximum concentration of Gd(III) in the lymph node after opacification with G6 Gd dendrimer was 466±145 ppmGd (2.9±0.9 mMGd) at 24 minutes post-injection and 427±130 ppmGd (2.7±0.8 mMGd) at 36 minutes. However, no significant (<0.02 mMGd/3.2 ppm) increased signal intensity was detected in the tissues adjacent to the axillary lymph node. Accumulation of G6 agent in the lateral thoracic lymph nodes was also found in three of five mice. The concentrations of Gd contained within the G6 dendrimer conjugate within the lateral thoracic lymph nodes (0-441 ppm, 0-2.7 mMGd; 134±154 ppmGd/0.8±1.0 mMGd, for average and standard deviation, 3 values below the lower detection limit were calculated as 0 mMGd) were lower and accumulated more slowly than in the axillary lymph nodes in the same mice. These results are consistent with a previous study that the predominant lymphatic drainage from the mouse breast is to the axillary lymph nodes. The G6 agent can be delivered to the axillary lymph node in sufficient quantity to visualize the lymph nodes by MRL even with an 11.5-fold dilution of the amount of injected agent used here. Since the lowest level of detection is 0.02 mMGd for G6 agent with this 1.5T MRI system as described above (FIGS. 21 and 22), the axillary lymph node contained more than 145-fold greater Gd(III) concentration than its adjacent soft tissue.

From the validation study using three different flip angles, although T1 values were too short to accurately measure the concentration of Gd ions, since this method was reliable to measure T1 value as small as 50 ms (corresponding to 2 mM for the G6 agent), the concentration of Gd ions was much more than 2 mM in 3 of 7 axillary lymph nodes at 24 minutes post-injection. Therefore, although both methods were not perfectly quantitative, the Gd concentration obtained from T1 measurement supported the data obtained using the signal intensity measurement compared with phantoms. Since the lowest level of detection was 3.2 ppm/0.02 mMGd with the phantom method and 0.8 ppm/0.005 mMGd with the T1 measurement method for the G6 agent, the axillary lymph nodes were found to contain a 145-1200-fold greater Gd(III) concentration than adjacent soft tissue.

Validation of Contrast Agent Delivery to Lymph Nodes with Scattered Tumor Metastasis

In order to validate the quantity of dendrimer conjugate delivery into a lymph node with scattered tumor metastasis, seven micro-metastasis model mice were evaluated. Mice were imaged when tumors of 4-7 mm developed in the mammary pad, usually at 15 days post-injection. Two of seven left axillary lymph nodes were palpable. The other five left axillary lymph nodes were visually normal in shape. Images were taken with 3D-fast spoiled gradient echo with 36 slice encoding steps, scan time 4 min 49 seconds at 6, 12 18, 24, 30, and 36 min post-injection of each contrast agent. All 7 axillary nodes were resected immediately after MRI scans and fixed with 10% formalin. Histology on a center slice of each sample was examined with hematoxylin and eosini (H-E) stating using a light microscope (×10-×400, Olympus, Melville, N.Y.). The scattered eosinophilic PT-18 cells were demonstrated in 6 of 7 lymph nodes on histological specimens. Therefore, only 6 mice with tumor-bearing lymph nodes were included in the data analysis.

Serial dynamic MR lymphangiograms of small PT-18 xenograft/lymph node metastasis model mice were obtained with 3D-fastSPGR 12, 24, 36, and 48 min after injection of the G6 contrast agent (40 mMGd, 6400 ppm). Gd(III) in the left axillary lymph node of each mouse was calculated with both phantom and T1 measurement methods, as described above.

High concentrations of G6 dendrimer conjugate can be found in lymph nodes containing metastatic disease. The axillary lymph nodes with small metastasis contained 402±134 ppmGd (2.5±0.8 mMGd) of Gd(III) in the G6 agent at 24 minutes post-injection and this was not significantly different from non-metastatic lymph nodes (p=0.42). However, in two of the six involved lymph nodes, an inhomogeneous low signal intensity near the center of the lymph node was observed, corresponding to relatively large micro-metastic cell foci, which might contain a slightly lower concentration than in the other four lymph nodes. The concentration was nonetheless sufficient for visualization by MRI.

Sentinel lymph node imaging is routine in breast cancer management. Preoperative lymphoscintigraphy with Tc-99m human serum albumin and intraoperative gamma probes are used to localize sentinel nodes. However, MRI has a number of potential advantages compared with lymphoscintigraphy, including higher spatial resolution enabling depiction of lymphatic channels, higher temporal resolution, three-dimensional images, and the absence of ionizing radiation exposure. Since the timing of the sentinel lymph node visualization varies in individual mice even with the same strain and same age, the timing of visualization in humans may have more variation. Therefore, dynamic MRL may find a role in the identification of a sentinel lymph node as well as the diagnosis of metastasis.

Suitable lymphatic imaging agents are typically small enough to enter the lymphatic vessels, yet large enough to be retained within the lymphatics and not leak from the capillary vessels. Lymphographic contrast agents are typically at least 4 nm in diameter to enable efficient retention within the lymphatics. Molecules smaller than 4 nm in diameter tend to diffuse into the surrounding tissue, resulting in poor signal to background ratios. Larger molecules, on the other hand, diffuse more slowly from the interstitial space and thus accumulate more slowly in the sentinel node, requiring a longer imaging time for visualizing nodes. Currently, there are two different methods for detecting sentinel nodes of the breast cancer; peri-tumoral subdermal and areolal intradermal injection. Since the areola of the mice were too small to inject contrast agents, peri-tumoral subdermal injection was used to detect the lymphatic drainage from the breast cancer. Therefore, the larger diameter G8 agent (−42 nm), used intradermally, may be too large for rapid uptake by lymphatic vessels from subdermal space of the breast tissue. In contrast, the G6 contrast agent is retained in the lymphatic vessels, resulting in efficient enhancement of lymphatic vessels and lymph nodes. Both the G2 and G4 agents and Gd-DTPA, which showed significantly lower signal (P<0.01) in the lymph node and higher signal in the adjacent muscle than G6 and G8, do not stay as well within the lymphatic vessels due to their small size, resulting in convection away from the injection site and only minimal enhancement in the lymph nodes. Moreover, rapid uptake of Gd-DTPA, even when used at a triple dose compared with the dose used for all other macromolecular agents, did not help to achieve good enough contrast between a target lymph node and the surrounding tissue at 3 min post-injection (data not shown).

The G6 dendrimer conjugate is a suitable choice among a number of similar macromolecular MR contrast agents for performing MRL and preserves a high signal even in the presence of lymph node metastases. The G6 dendrimer conjugate was found to have the most rapid and intense enhancement of all of the lymphatic agents tested. The PAMAM dendrimers are identical in all chemical respects except molecular diameter, thus allowing the effect of molecular size to be isolated from other molecular features (charge, hydrophilicity, etc.). Although prior reports have demonstrated differences in the in vivo pharmacokinetics of macromolecular contrast agents based on molecular weight alone, these agents have also differed in their chemical properties, making it difficult to distinguish the effect of molecular size from other chemical properties of the molecules. However, the body recognizes and processes nano-size molecules differently depending on their size; differences of only about 3 nm in diameter dramatically affect pharmacokinetics when these agents are injected intravenously. When these same agents are injected into interstitial tissues, parallel differences in lymphatic transport can be seen.

Slower uptake into the lymphatics is seen with larger nano-particles, such as ultra-small particles of iron oxide (USPIO), which are about 30 nm in diameter. USPIOs have been employed as lymphatic agents after intravenous injection because they are engulfed by macrophages in the serum, which travel to systemic lymph nodes. The USPIO employed in this study did not depict the lymphatic vessels even at high doses (data not shown).

DAB-G5 and Gadomer-17, which are theoretically less hydrophilic than the PAMAM dendrimer-based agents, showed the axillary lymph nodes with better contrast than PAMAM dendrimer-based agents of similar sizes (equivalent to G4 and G2 dendrimers), although neither agent depicted lymphatic vessels. Unless agents are trapped by any cells, sooner or later all agents, which were injected subcutaneously, should flow from the lymphatic system to the blood circulation. Gadomer-17 rapidly cleared from the body when injected intravenously, and is an alternative agent to visualize and treat lymph nodes with this method.

Nevertheless, since the detection limit of metastatic nodules by mMRML remains at ˜100 μm with clinical MRI systems, this method is less likely to detect small clusters of malignant cells. For such micro-metastases additional methods are useful for both diagnosis and treatment. Because G6 dendrimer conjugate is an effective delivery mechanism to the lymphatics draining tumors, it serves as a target or carrier for therapy to regional lymph nodes as well. For instance, the accumulation of gadolinium within the lymph nodes with relatively little background accumulation permits the use of NCT to selectively treat regional lymph nodes.

A major current limitation of Gd-NCT is that 400 ppm of natural Gd(III), which equates to about 64 ppm of ¹⁵⁷Gd, appears to be needed for efficient cell killing. This concentration can be difficult to achieve using conventional intravenous routes of delivery. Therefore, most of in vivo studies have been performed with intra-tumoral or intra-arterial injection of agents. However, the inventors have now shown that comparable concentrations of gadolinium were achieved within the lymph nodes.

Previous reports have shown that using an intraperitoneal injection concentrations of 162 ppm Gd(III) within disseminated tumor tissue were achieved using a G6-avidin system. However, using the newly described method, even greater Gd(III) concentrations were achieved within the sentinel lymph node by intra-mammary gland injection of the G6 nano-size contrast agent. The amount of local lymphatic drainage correlated well with the number of cancer cells migrating to the draining lymph node. Moreover, the concentration of G6 agent within the sentinel lymph node was monitored by MRL. No toxicity of any of the PAMAM G8, G6 and G4 agents in mice was observed with 25 times as much as the highest dose (40 mM/20 μL) used in this study. Taken together, peri-tumoral injection of a nano-size G6 agent provides an imaging method to detect sentinel lymph nodes and direct Gd-NCT to primary tumors and their sentinel lymph nodes.

Sentinel node imaging is able to identify lymph nodes and also identify metastatic disease within them. Metastases might obstruct lymphatic flow leading to collateral lymphatic circulation which may not allow the agent into the sentinel lymph node. However, if metastatic lymph nodes are larger than the detectable size with MRL they are clinically obvious with conventional imaging. Therefore, this technique is believed to be of particular benefit to patients with micro-metastasis of cancer in the lymph nodes where lymph nodes would not only be easily identified but also amenable to Gd-NCT.

In this study, the gadolinium concentrations within the lymph nodes were estimated based on both phantom studies and T1 measurements but could not be accurately calculated based on relaxivity in the lymph node tissue. Since the tissue in the lymph node is not as homogenous as serum phantoms, this inhomogeneity can change the relaxivity of G6 and affect to the T1 signal intensity due to the susceptibility artifact. However, since the values obtained with two different methods were consistent, it is believed that these MRI methods provide a close approximation to the actual concentrations of Gd(III) expected within the lymph nodes.

In conclusion, among all the agents tested, the Gd(III) labeled G6 dendrimer best depicted the lymph nodes and lymphatic channels. MR lymphangiography with interstitial injections can localize sentinel nodes by accumulating sufficient concentrations of Gd(III) to allow MR imaging. These same concentrations of Gd(III) can be utilized to direct Gd-NCT to effectively treat lymph node metastases, by enabling irradiation of tissues rich in Gd(III) labeled G6 dendrimer for NCT.

### EXAMPLE 5

### MRI

MRI is a technique that allows whole body in vivo imaging in three dimensions at high resolution. In MRI, a static magnetic field is applied to the object of interest while simultaneously or subsequently applying pulses of radio frequency (RF) to change the distribution of the magnetic moments of protons in the object. The change in distribution of the magnetic moments of protons in the object from their equilibrium (normal) distribution to a non-equilibrium distribution and back to the normal distribution (via relaxation processes) constitute the MRI signal.

The longitudinal relaxation time, T₁, is defined as the time constant of the exponential recovery of proton spins to their equilibrium distribution along an applied magnetic field after a disturbance (e.g. a RF pulse). The transverse relaxation time, T₂, is the time constant that describes the exponential loss of magnetization in a plane transverse to the direction of the applied magnetic field, following a RF pulse that rotates the aligned magnetization into the transverse plane. Magnetic resonance (MR) contrast agents assist this return to a normal distribution by shortening T₁ and/or T₂ relaxation times.

Signal intensity in biological MRI depends largely on the local value of the longitudinal relaxation rate (1/T₁), and the transverse relaxation rate (1/T₂) of water protons. Contrast agents will increase 1/T₁ and/or 1/T₂, depending on the nature of the agent and the strength of the applied field. MRI pulse sequences that emphasize changes in 1/T₁ are referred to as T₁-weighted and those that emphasize changes in 1/T₂ are referred to as T₂-weighted. MR contrast agents that include gadolinium (III) ions increase both 1/T₁ and 1/T₂, and are primarily used with T₁-weighted imaging sequences, since the relative change in 1/T₁ in tissue is typically much greater than the change in 1/T₂. Iron particles, by contrast, provide larger relative changes in 1/T₂, and are best visualized in a T₂-weighted image.

Advances in MRI have tended to favor T₁ agents such as gadolinium (III) based contrast agents. Faster scans with higher resolution require more rapid RF pulsing, and can lead to loss of the MRI signal through saturation effects. T₁ agents relieve this saturation and restore signal intensity by stimulating relaxation of nuclear spins between RF pulses. Furthermore, T₁ agents are compatible with image guided surgical procedures such as needle biopsy, as objects inserted into a subject's body will appear in a T₁ image.

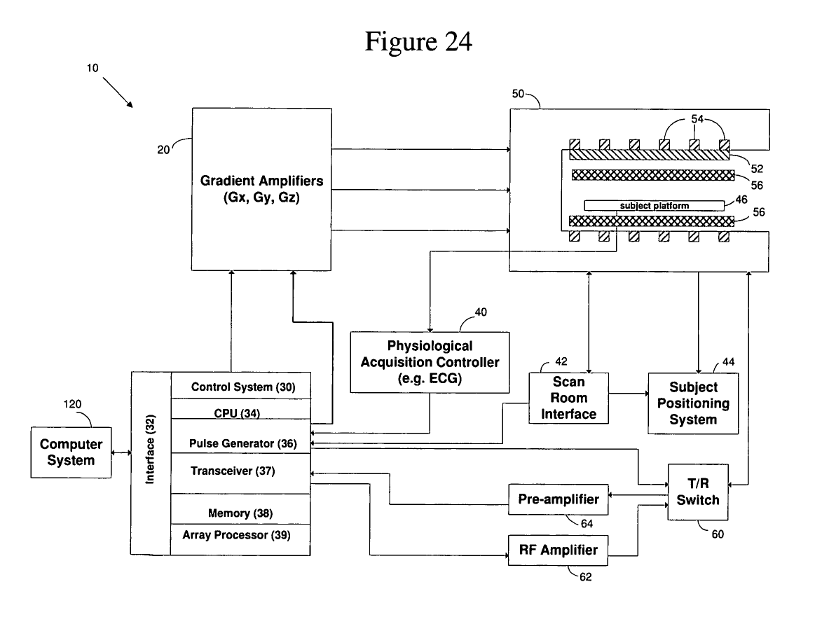

An exemplary MRI system is illustrated in FIG. 24. Referring to FIG. 24, the major components of a MRI system 10 that may be used to practice the disclosed methods are shown. The operation of the system is controlled by computer system 120. The computer system 120 includes a number of modules that communicate with each other, and with control system 30, through interface 32.

The control system 30 includes a set of modules connected together by an interface 32, and also connected to computer system 120 through interface 32. These modules include a CPU module 34. A pulse generator module 36 operates the system components to carry out the desired scan sequence and produces data which indicates the timing, strength and shape of the RF pulses produced, and the timing and length of the data acquisition window. The pulse generator module 36 connects to a set of gradient amplifiers 20, to indicate the timing and shape of the gradient pulses that are produced during the scan. The pulse generator module 36 also receives subject data from a physiological acquisition controller 40 that receives a signal from one or more sensors connected to the subject, such as an ECG signal from electrodes attached to the subject. The pulse generator module 36 also connects to a scan room interface circuit 42 that receives signals from various sensors associated with the condition of the patient and the magnet system. It is also through the scan room interface circuit 42 that a subject positioning system 44 receives commands to move the subject on subject platform 46 to the desired position for the scan.

The gradient waveforms produced by the pulse generator module 36 are applied to the gradient amplifier system 20 having Gx, Gy, and Gz amplifiers. Each gradient amplifier excites a corresponding gradient coil in an assembly designated 52. The gradient coil assembly 52 forms part of a magnet assembly 50 which includes a polarizing magnet 54 and a whole-body RF coil 56. Although not shown, additional coils may be used to provide more detailed images of a particular anatomical location within or on a subject. For example an external coil such as a breast coil, head coil, cardiac coil, CTL coil, shoulder coil, or torso-pelvis coil is used (these types of coils and others are available from GE Medical Systems, Milwaukee, Wis.). In a particular embodiment, a breast coil is located over a female subject's mammary glands to provide more detailed images of the mammary tissue. A transceiver module 37 in the control system 30 produces pulses that are amplified by a RF amplifier 62 and coupled to the RF coil 56 by a transmit/receive switch 60. The resulting signals radiated by the excited nuclei in the patient may be sensed by the same RF coil 56 and coupled through the transmit/receive switch 60 to a preamplifier 64. The amplified NMR signals are demodulated, filtered, and digitized in the receiver section of the transceiver 37. The transmit/receive switch 60 is controlled by a signal from the pulse generator module 36 to electrically connect the RF amplifier 62 to the coil 56 during the transmit mode and to connect the preamplifier 64 during the receive mode. The transmit/receive switch 60 also enables a separate RF coil (for example, a surface coil) to be used in either the transmit or receive mode.

The following is a brief description of the acquisition and storage of MR data. The NMR signals picked up by the RF coil 56 are digitized by the transceiver module 37 and transferred to a memory module 38 in the system control 32. When a scan is completed, an array of raw k-space data has been acquired in the memory module 38. This raw k-space data is rearranged into separate k-space data arrays for each image to be reconstructed, and each of these is input to an array processor 39 which operates to Fourier transform the data into an array of image data. This image data is conveyed through interface 32 to the computer system 120, where it may be stored and/or further processed using methods known to those skilled in the art.

### EXAMPLE 6

### Dendrimer Conjugates Having a Metal Chelate

Dendrimer-based contrast agents with a metal chelate may be prepared by reacting a surface group of a dendrimer with the reactive group of a bifunctional chelating agent and then reacting the metal chelating group of the bifunctional chelating agent with a metal ion. Alternatively, a metal ion is reacted with the metal chelating group of the bifunctional chelating agent prior to reacting the reactive group of the bifunctional chelating agent with a surface groups of the dendrimer. Metal chelation is typically carried out in solution, and desirably avoids the use of strong acids or bases. In particular embodiments, a dendrimer, such as generation 2 polylysine, DAB-G4D, DAB-G5D, DAB-G6D, DAB-G7D, DAB-G8D, PAMAM-G4D, PAMAM-G5D, PAMAM G6D, PAMAM-G7D, or PAMAM-G8D is reacted with 1B4M and gadolinium ions (in either order as discussed below) to provide dendrimer conjugates suitable for lymphatic system imaging or delivery of an anti-tumor agent, such as for NCT.

Thus, in one aspect, dendrimer conjugates suitable for lymphatic system imaging or delivery of an anti-tumor agent, such as for NCT include, Gadomer-17, DAB-G4, DAB-G5, DAB-G6, DAB-G7, DAB-G8, PAMAM-G4, PAMAM-G5, PAMAM-G6, PAMAM-G7, and PAMAM-G8, which all are 1B4M conjugates with chelated Gd³⁺ ions. In more particular examples, dendrimer conjugates for use as lymphatic system contrast or NCT agents include DAB-G5, PAMAM-G6 and PAMAM-G8. In still more particular examples, a PAMAM-G6 dendrimer conjugate is used for lymphatic system imaging or NCT.

Dendrimer conjugates may also be used for delivery of an anti-tumor agent, such as for NCT. In particular examples, dendrimer conjugates which may be useful in NCT, particularly for NCT of the lymphatic system, include DAB-G5, Gadomer-17, PAMAM-G6, PAMAM-G7, and PAMAM-G8.

Table 3 compares some properties of some particular dendrimer conjugates, Gadomer-17 and the simple gadolinium chelate GPDM.

TABLE 3
Comparison of Example Contrast Agents

| | | | Approximate | |
|---|---|---|---|---|
| Contrast | Approximate | | Core MW | |
| Agent | MW (kD) | Gd atoms | (kD) | Dendrimer Type |
| PAMAM-G4 | 58 | 64 | 14.2 | PAMAM |
| PAMAM-G5 | 117 | 128 | 29 | PAMAM |
| PAMAM-G6 | 235 | 256 | 58 | PAMAM |
| PAMAM-G7 | 470 | 512 | 116 | PAMAM |
| PAMAM-G8 | 960 | 1024 | 233 | PAMAM |
| DAB-G5 | 51 | 128 | n/a | DAB |
| Gadomer-17 | 30 | 24 | n/a | Aromatic Ring |
| | | | | Core Dendrimer |
| Gd-DTPA- | 0.94 | 1 | n/a | n/a |
| dimeglumine | | | | |

n/a—not available or not applicable

The disclosed dendrimer conjugates exhibit a range of properties that permit detailed and selective imaging or NCT of particular components (or functions) of the lymphatic system (such as lymphatic vessels, lymph nodes and flow of lymphatic fluid). For example, PAMAM-G8 exhibits lymphotropic behavior (accumulation in the lymph system) and minimal leakage out of the lymphatic vessels, which aids in the visualization or NCT of both thick and thin lymphatic vessels. In contrast, PAMAM-G4 and DAB-G5 tend to accumulate in the lymph nodes rather than the vessels, and provides detailed visualization of these structures and the opportunity to perform NCT on the lymph nodes. PAMAM-G4 has a short survival in the blood circulation due to a rapid renal excretion without significant retention in other organs. PAMAM-G6 has an intermediate survival period in the lymph system, and is particularly suitable for dynamic imaging of the lymph system (for example, for following lymphatic flow) and for NCT of the lymph system.

Additional dendrimers may be used to provide dendrimer conjugates that can be utilized in the disclosed methods. For example, polyakylenimine dendrimers and PAMAM dendrimers having different initiator cores, but similar molecular weights (within about 25%, for example within 15%, 10% or 5% of the MW) to those dendrimers specifically disclosed may be utilized. Such dendrimers also may be synthesized according to the methods disclosed in Womer et al., Angewandte Chemie, Int. Ed.; 32: 1306-1308, 1993. Similar methods, and in particular, methods for making polypropylenimine dendrimers having various initiator cores, such as ammonia, ethylenediamine, propylenediamine, diaminobutane and other polyamines such as tris-aminoethylamine, cyclene, hexaazacyclooctadecane, 1,5 diaminopentane, ethylenetriamine, triethylenetetramine, 1,4,8,11-tetraazaundecane, 1,5,8,12-tetraazaundodecane, and 1,5,9,13-tetraazatridecan are discussed by De Brabander-van den Berg et al., Angewandte Chemie, Int. Ed.; 32: 1308, 1993. Typically, the surface of the polypropylenimine dendrimer will have one or more amino groups. However, some or all of the surface amino groups may be modified, for example, to provide other reactive groups or charged, hydrophilic, and/or hydrophobic groups such as carboxylate, hydroxyl and alkyl groups on the surface. Similar schemes may be used to synthesize polybutylenimine and higher polyalkylenimine dendrimers. Additional information regarding the synthesis of a variety of dendrimers with branches formed from vinyl cyanide units is provided in PCT Publication WO 93/14147.

PAMAM dendrimers also may be synthesized from a variety of core molecules (e.g., those described above for DAB dendrimers) according to the methods disclosed in U.S. Pat. No. 5,338,532. Dendrimers having other surface groups, such as carboxylate and hydroxyl, also are available commercially (Aldrich, Milwaukee, Wis.) or may be provided by the methods disclosed in U.S. Pat. No. 5,338,532.

The metal chelate in a dendrimer conjugate may be a complex of a metal ion and a metal chelating group (a group of atoms that serves to bind the metal ion). Examples of metal chelating groups include natural and synthetic amines, porphyrins, aminocarboxylic acids, iminocarboxylic acids, ethers, thiols, phenols, glycols and alcohols, polyamines, polyaminocarboxylic acids, polyiminocarboxylic acids, aminopolycarboxylic acids, iminopolycarboxylic acids, nitrilocarboxylic acids, dinitrilopolycarboxlic acids, polynitrilopolycarboxylic acids, ethylenediaminetetracetates, diethylenetriaminepenta or tetraacetates, polyethers, polythiols, cryptands, polyetherphenolates, polyetherthiols, ethers of thioglycols or alcohols, polyaminephenols, all either acyclic, macrocyclic, cyclic, macrobicyclic or polycyclic, or other similar ligands which produce stable metal chelates or cryprates (including sepulchrates, sacrophagines, and crown ethers).

Specific examples of metal chelating groups include diethylenetriaminepentaacetic acid (DTPA), 1,4,7,10-tetraazacyclododecanetetraacetic acid (DOTA), 1,4,7,10-tetraazacyclododecane-1,4,7-triacetic acid (DO3A), 1-oxa-4,7,10-triazacyclododecane-triacetic acid (DOXA), 1,4,7-triazacyclononanetriacetic acid (NOTA), 1,4,8,11-tetraazacyclotetradecanetetraacetic acid (TETA), DOTA-N(2-aminoethyl)amide and DOTA-N-(2-aminophenethyl)amide, BOPTA, HP-DO3A, DO3MA, 1B4M and various derivatives and combinations thereof. Additional examples of metal chelating groups have been described by Caravan et al. (Caravan et al., Chem. Rev. 99: 2293-2352, 1999). Since release of metal ions from such chelating groups can be dangerous to a subject, it is advantageous to select a metal chelating group that tightly binds a metal ion. Therefore, a high stability constant for the metal chelate is desired.

The reactive group of a bifunctional chelating agent is a group of atoms that will undergo a reaction with a surface group of a dendrimer to form a bond, such as a covalent bond. Examples of reactive groups include carboxylic acid groups, diazotiazable amine groups, N-hydroxysuccinimidyl, esters, aldehydes, ketones, anhydrides, mixed anhydrides, acyl halides, maleimides, hydrazines, benzimidates, nitrenes, isothiocyanates, azides, sulfonamides, bromoacetamides, iodocetamides, carbodiimides, sulfonylchlorides, hydroxides, thioglycols, or any reactive group known in the art as useful for forming conjugates. If the dendrimer is a DAB-Am dendrimer, the reactive group may be a functional group capable of undergoing reaction with an amino group of the DAB-Am dendrimer.

Specific examples of bifunctional chelating agents include bifunctional diethylenetriaminepentaacetic acid (DTPA) derivatives such as those disclosed in U.S. Pat. No. 5,434,287. Other examples include polysubstituted diethylenetriaminepentaacetic acid chelates such as those described in U.S. Pat. No. 5,246,692. Bifunctional chelating agents comprising 1,4,7,10-Tetraazacyclododecane-N,N′,N″,N′″-tetraacetic acid (DOTA) and its derivatives are also useful. Examples of bifunctional DOTA derivatives are provided in U.S. Pat. No. 5,428,154 to Gansow et al. and references therein. A particular example of a bifunctional chelating agent is 2-(p-isothiocyanatobenzyl)-6-methyl-diethylenetriaminepentaacetic acid (1B4M).

Additional examples of bifunctional chelating agents and metal chelating groups may be found in U.S. Pat. Nos. 5,292,868, 5,364,613, 5,759,518, 5,834,020, 5,874,061, 5,914,095, 5,958,373, 6,045,776, 6,274,713; PCT Publications WO 95/17451 and WO 95/09564; U.S. Patent Application Publication US2002/0004032; European Patent Application EP 0882454; and European Patent Specifications EP 0416033 and EP 0497926.

Metals ions of the metal chelates may be paramagnetic ions if the imaging agent is to be used as a MRI contrast agent. Suitable ions include ions of metals having atomic numbers of 22-29 (inclusive), 42, 44 and 58-70 (inclusive) and combinations thereof. In particular embodiments, the metal ions have an oxidation state of 2 or 3. Examples of such metal ions are chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III) and ytterbium (III), and combinations thereof. Particular examples of useful ions for MRI include the paramagnetic ions of gadolinium, dysprosium, cobalt, manganese, and iron. In a particular disclosed embodiment, the metal ion is a Gd (III) ion.

If the macromolecular imaging agent is to be used as an X-ray contrast agent (such as for CT), the metal ion may be selected from the ions of W, Bi, Hg, Os, Pb, Zr, lanthanides, and combinations thereof. If a combined MRI/X-ray contrast agent is desired, the metal ion may be selected from the paramagnetic lanthanide ions. If a scintographic imaging agent is desired, the metal may be radioactive, such as the radioactive isotopes of Gd, In, Tc, Y, Re, Pb, Cu, Ga, Sm, Fe, or Co.

If the dendrimer chelate is to be used as a NCA, the metal may be a NCE, such as gadolinium, including ¹⁵⁵Gd or ¹⁵⁷Gd. Other metals having a suitably large neutron capture cross section may be used.

In some embodiments, the methods include administering a dendrimer conjugate to a subject where the metal chelating group of the dendrimer conjugate is diethylenetriaminepentaacetic acid (DTPA), 1,4,7,10-tetraazacyclododecanetetraacetic acid (DOTA), 1,4,7,10-tetraazacyclododecane-1,4,7-triacetic acid (DO3A), 1-oxa-4,7,10-triazacyclododecane-triacetic acid (DOXA), 1,4,7-triazacyclononanetriacetic acid (NOTA), 1,4,8,11-tetraazacyclotetradecanetetraacetic acid (TETA), DOTA-N-(2-aminoethyl)amide and DOTA-N-(2-aminophenethyl)amide, BOPTA, HP-DO3A, DO3MA, 2-(p-isothiocyanatobenzyl)-6-methyl-diethylenetriaminepentaacetic acid (1B4M), or derivatives and combinations thereof. The metal chelate may comprise an ion of a metal having an atomic number of 22-29, 42, 44, 58-70 or combinations thereof. In particular embodiments, the ion is a chromium (III) ion, manganese (II) ion, iron (II) ion, iron (III) ion, cobalt (II) ion, nickel (II) ion, copper (II) ion, praseodymium (III) ion, neodymium (III) ion, samarium (III) ion, gadolinium (III) ion, terbium (III) ion, dysprosium (III) ion, holmium (III) ion, erbium (III) ion, ytterbium (III) ion or a combination of such ions. In particular embodiments, the dendrimer conjugate is a Gd-1B4M conjugate and is DAB-G5, DAB-G6, DAB-G7, DAB-G8, PAMAM-G5, PAMAM G6, PAMAM-G7, or PAMAM-G8.

We claim:

1. A method of treating a tumor in the lymphatic system, comprising:
administering to a subject who has been identified as having a tumor a dendrimer conjugate that selectively concentrates in the lymphatic system of the subject, wherein the dendrimer conjugate comprises a generation 5 DAB or generation 3-10 PAMAM dendrimer conjugated to an amount of an anti-tumor agent effective to treat the identified tumor, but the dendrimer conjugate does not include a tumor specific targeting agent; and
treating the tumor with the anti-tumor agent.

2. The method of claim 1, wherein the anti-tumor agent is a cytotoxic agent that acts on a tumor without requiring activation.

3. The method of claim 1, wherein the cytotoxic agent is an activatable cytotoxic agent, and the cytotoxic agent is activated by application of physical energy from outside the body.

4. The method of claim 3, wherein the activatable cytotoxic agent comprises an agent activatable by a neutron beam, and the agent is activated by selectively directing a neutron beam at the lymphatic system of the subject.

5. The method of claim 4, wherein the activatable cytotoxic agent further comprises an imaging agent that is activatable by the neutron beam to be a cytotoxic agent.

6. The method of claim 4, wherein the dendrimer conjugate further comprises an imaging agent, and selectively directing a neutron beam at the lymphatic system of the subject comprises imaging the dendrimer conjugate in the lymphatic system, and directing the neutron beam at the imaged dendrimer conjugate.

7. The method of claim 6, wherein imaging the dendrimer conjugate comprises determining whether a tumor is present in an imaged lymph node, and directing the neutron beam at the imaged lymph node if the tumor is detected in the lymph node.

8. The method of claim 6, wherein the dendrimer conjugate comprises a gadolinium contrast agent that also acts as an activatable cytotoxic agent.

9. The method of claim 1, further comprising identifying a subject as having a tumor.

10. The method of claim 9, wherein identifying the subject as having a tumor comprises identifying the subject as having the tumor prior to administering the dendrimer conjugate.

11. The method of claim 9, wherein identifying the subject as having a tumor comprises identifying the subject as having a tumor that spreads through the lymphatic system, and administering the dendrimer conjugate comprises treating potential micrometastatic disease.

12. The method of claim 1, wherein administering the dendrimer conjugate comprises injecting the agent into or near a tumor.

13. The method of claim 1, wherein administering the dendrimer conjugate comprises injecting the agent directly into the afferent lymphatic system of a tumor.

14. The method of claim 1, wherein administering the dendrimer conjugate comprises selecting the dendrimer conjugate which selectively concentrates in a component of the lymphatic system in which the tumor is located.

15. The method of claim 1, wherein administering the dendrimer conjugate comprises peritumoral injection, intratumor injection, or intradermal injection.

16. The method of claim 1, wherein the dendrimer conjugate has a diameter of about 4 nanometers to about 15 nanometers.

17. The method of claim 1, wherein the dendrimer conjugate comprises a generation 4-8 PAMAM dendrimer.

18. The method of claim 1, wherein the dendrimer conjugate comprises a generation 6-8 PAMAM dendrimer.

19. A method of inhibiting metastatic disease in a subject, comprising:
identifying a subject as having a tumor that spreads through the lymphatic system;
administering a dendrimer conjugate in the lymphatic system of the subject who has been identified as having a tumor, wherein the dendrimer conjugate comprises a generation 5 DAB or generation 3-10 PAMAM dendrimer and is capable of selectively concentrating in lymphatic structures and is conjugated to an amount of a gadolinium imaging agent that can be activated by a neutron beam to treat the identified tumor, wherein the dendrimer conjugate does not include a tumor specific targeting agent;
imaging the lymphatic system to detect the gadolinium imaging agent in the lymphatic system; and
selectively activating the gadolinium imaging agent by directing a neutron beam at a portion of the imaged lymphatic system, thereby treating metastatic disease that may be in the lymphatic system.

20. A method of inhibiting metastatic disease in a subject, comprising:
selecting a dendrimer conjugate having a diameter of about 4 nanometers to about 15 nanometers for administration to a subject who has been identified as having a tumor;
administering the dendrimer conjugate in the lymphatic system of the subject, wherein the dendrimer conjugate comprises a generation 5 DAB or generation 3-10 PAMAM dendrimer and is capable of selectively concentrating in lymphatic structures and is conjugated to an amount of a gadolinium imaging agent that can be activated by a neutron beam to treat the identified tumor;
imaging the lymphatic system to detect the gadolinium imaging agent in the lymphatic system; and
selectively activating the gadolinium imaging agent by directing a neutron beam at a portion of the imaged lymphatic system, thereby treating metastatic disease that may be in the lymphatic system.

21. The method of claim 20, wherein the dendrimer conjugate does not include a tumor specific targeting agent.
